(19) United States
(12) Patent Application Publication
(10) Pub. No.: US 2025/0051465 A1
(43) Pub. Date: Feb. 13, 2025

(54) ANTIGEN-BINDING PROTEINS THAT ACTIVATE THE LEPTIN RECEPTOR

(71) Applicant: Regeneron Pharmaceuticals, Inc., TARRYTOWN, NY (US)

(72) Inventors: Jesper GROMADA, Scarsdale, NY (US); Panayiotis STEVIS, West Orange, NJ (US); Judith ALTAREJOS, Chappaqua, NY (US)

(21) Appl. No.: 18/810,454

(22) Filed: Aug. 20, 2024

Related U.S. Application Data
(63) Continuation of application No. 17/539,034, filed on Nov. 30, 2021.
Application No. 17/539,034 is a division of application No. 16/593,761, filed on Oct. 4, 2019, now abandoned.
Application No. 16/593,761 is a division of application No. 16/007,812, filed on Jun. 13, 2018, now Pat. No. 10,618,968.
Application No. 16/007,812 is a division of application No. 15/290,967, filed on Oct. 11, 2016, now Pat. No. 10,023,644.
(60) Provisional application No. 62/240,021, filed on Oct. 12, 2015.
(60) Provisional application No. 62/359,757, filed on Jul. 8, 2016.
(60) Provisional application No. 62/375,495, filed on Aug. 16, 2016.
(60) Provisional application No. 62/393,143, filed on Sep. 12, 2016.

(51) Int. Cl. C07K 16/28; A61K 39/00; A61K 39/395

(52) U.S. Cl. CPC C07K 16/2869; A61K 39/395; A61K 2039/505; C07K 2317/21; C07K 2317/33; C07K 2317/34; C07K 2317/565; C07K 2317/75; C07K 2317/92

(57) ABSTRACT

The present invention provides antibodies and antigen-binding fragments of antibodies that bind to leptin receptor (LEPR), and methods of using the same. According to certain embodiments, the invention includes antibodies and antigen-binding fragments of antibodies that bind LEPR and activate LEPR signaling. In other embodiments, the invention includes antibodies and antigen-binding fragments of antibodies that bind to LEPR and enhance sensitization of LEPR to an antigen. In certain embodiments, the invention includes antibodies and antigen-binding fragments of antibodies that bind LEPR in the presence and absence of leptin. In certain embodiments, the invention includes antibodies and antigen-binding fragments of antibodies that induce signaling in cells expressing LEPR mutants that otherwise exhibit defective or impaired signaling in the presence of leptin. The antibodies and antigen-binding fragments of the present invention are useful for the treatment of lipodystrophies and other diseases and disorders associated with or caused by leptin deficiency or leptin resistance.

This application is a continuation of U.S. patent application Ser. No. 17/539,034, filed on Nov. 30, 2021, which is a divisional of U.S. patent application Ser. No. 16/593,761, filed on Oct. 4, 2019, which is a divisional application of U.S. patent application Ser. No. 16/007,812, filed on Jun. 13, 2018, now U.S. Pat. No. 10,618,968, which is a divisional of U.S. patent application Ser. No. 15/290,967, filed Oct. 11, 2016, now U.S. Pat. No. 10,023,644, which claims the benefit of priority under 35 U.S.C. § 119 (e) of U.S. Provisional Application Nos. 62/240,021, filed Oct. 12, 2015; 62/359,757, filed Jul. 8, 2016; 62/375,495, filed Aug. 16, 2016, and 62/393,143, filed Sep. 12, 2016, the disclosures of which are herein incorporated by reference in their entireties.

### SEQUENCE LISTING

An official copy of the sequence listing is submitted concurrently with the specification electronically via Patent Center. The contents of the electronic sequence listing (10178US06_Sequence_Listing_ST26.xml; Size 163,840 bytes; and Date of Creation: Aug. 20, 2024) is herein incorporated by reference in its entirety.

### FIELD OF INVENTION

The present invention is related to antibodies and antigen-binding fragments of antibodies that bind human leptin receptor (LEPR), and therapeutic and diagnostic methods of using those antibodies.

### BACKGROUND

Leptin is a polypeptide hormone predominantly expressed by adipose tissue and is involved in the regulation of metabolism, energy balance and food intake. Leptin activity is mediated by interaction with, and signaling through, the leptin receptor. Leptin receptor, (also known as “LEPR,” “WSX,” “OB receptor,” “OB-R,” and “CD295”) is a single-pass transmembrane receptor of the class I cytokine receptor family with a large (818 amino acid) extracellular domain. Leptin deficiency, leptin resistance, and certain LEPR signaling-defective/signaling impaired mutations, are associated with obesity, type 2 diabetes, dyslipidemia, lipodystrophies, hepatic steatosis, non-alcoholic and alcoholic fatty liver diseases, severe insulin resistance, Leprechaunism/Donohue syndrome, Rabson-Mendenhall syndrome, and related complications. Therapeutic approaches to address leptin resistance, leptin deficiency, and hypoleptinemia (e.g., lipodystrophy) have mostly focused on the delivery of supplemental leptin or leptin analogues to affected individuals. Such approaches, however, have generally shown limited efficacy, particularly in leptin-resistant individuals, and are frequently associated with adverse side effects. Thus, a need exists in the art for alternative approaches to treating leptin resistance and other conditions associated with leptin deficiency or hypoleptinemia.

### BRIEF SUMMARY OF THE INVENTION

The present invention provides antibodies and antigen-binding fragments thereof that bind human leptin receptor (LEPR). The antibodies of the present invention are agonist antibodies; i.e., binding of the anti-LEPR antibodies of the invention to LEPR causes, inter alia, activation of leptin receptor signaling in cells. In certain embodiments, the antibodies of the present invention do not compete with leptin for binding to LEPR. The antibodies of the present invention are useful, e.g., for mimicking, substituting for, or supplementing the normal biological activity of leptin in a subject. The antibodies and antigen-binding fragments of the present invention are therefore useful in the therapeutic treatment of diseases and disorders associated with leptin resistance and leptin deficiency.

The antibodies of the invention can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., 2000, J. Immunol. 164:1925-1933).

Exemplary anti-LEPR antibodies of the present invention are listed in Tables 1 and 2 herein. Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-LEPR antibodies. Table 2 sets forth the nucleic acid sequence identifiers of the HCVRs, LCVRs, HCDR1, HCDR2 HCDR3, LCDR1, LCDR2 and LCDR3 of the exemplary anti-LEPR antibodies.

The present invention provides antibodies or antigen-binding fragments thereof that specifically bind LEPR, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies or antigen-binding fragments thereof that specifically bind LEPR, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies or antigen-binding fragments thereof that specifically bind LEPR, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-LEPR antibodies listed in Table 1. In certain embodiments, the HCVR/LCVR amino acid sequence pair is selected from the group consisting of SEQ ID NOs: 2/10, 18/10, 26/10, 34/10, 42/10, 50/10, 58/10, 66/10, 74/10, 82/90, 98/90, and 106/90.

The present invention also provides antibodies or antigen-binding fragments thereof that specifically bind LEPR, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind LEPR, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind LEPR, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind LEPR, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind LEPR, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind LEPR, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind LEPR, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-LEPR antibodies listed in Table 1. In certain embodiments, the HCDR3/LCDR3 amino acid sequence pair is selected from the group consisting of SEQ ID NOs: 8/16, 24/16, 32/16, 40/16, 48/16, 56/16, 64/72, 80/72 and 88/72.

The present invention also provides antibodies or antigen-binding fragments thereof that specifically bind LEPR, comprising a set of six CDRs (i.e., HCDR1, HCDR2, HCDR3, LCDR1, LCDR2, and LCDR3) contained within any of the exemplary anti-LEPR antibodies listed in Table 1. In certain embodiments, the HCDR1, HCDR2, HCDR3, LCDR1, LCDR2, and LCDR3 amino acid sequences set is selected from the group consisting of SEQ ID NOs: 4, 6, 8, 12, 14, 16; 20, 22, 24, 12, 14, 16; 28, 30, 32, 12, 14, 16; 36, 38, 40, 12, 14, 16; 44, 46, 48, 12, 14, 16; 52, 54, 56, 12, 14, 16; 60, 62, 64, 12, 14, 16; 66, 68, 70, 12, 14, 16; 76, 78, 80, 12, 14, 16; 84, 86, 88, 92, 94, 96; 100, 102, 104, 92, 94, 96; 106, 108, 110, 92, 94, 96.

In a related embodiment, the present invention provides antibodies, or antigen-binding fragments thereof that specifically bind LEPR, comprising a set of six CDRs (i.e., HCDR1, HCDR2, HCDR3, LCDR1, LCDR2, and LCDR3) contained within an HCVR/LCVR amino acid sequence pair as defined by any of the exemplary anti-LEPR antibodies listed in Table 1. For example, the present invention includes antibodies or antigen-binding fragments thereof that specifically bind LEPR, comprising the HCDR1, HCDR2, HCDR3, LCDR1, LCDR2, and LCDR3 amino acid sequences set contained within an HCVR/LCVR amino acid sequence pair selected from the group consisting of SEQ ID NOs: 2/10, 18/10, 26/10, 34/10, 42/10, 50/10, 58/10, 66/10, 74/10, 82/90, 98/90, and 106/90. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present invention also provides nucleic acid molecules encoding anti-LEPR antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1, HCDR2, HCDR3), wherein the HCDR1, HCDR2, HCDR3 amino acid sequence set is as defined by any of the exemplary anti-LEPR antibodies listed in Table 1.

The present invention also provides nucleic acid molecules encoding an LCVR, wherein the LCVR comprises a set of three CDRs (i.e., LCDR1, LCDR2, LCDR3), wherein the LCDR1, LCDR2, LCDR3 amino acid sequence set is as defined by any of the exemplary anti-LEPR antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 2, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-LEPR antibody listed in Table 1.

The present invention also provides recombinant expression vectors capable of expressing a polypeptide comprising a heavy or light chain variable region of an anti-LEPR antibody. For example, the present invention includes recombinant expression vectors comprising any of the nucleic acid molecules mentioned above, i.e., nucleic acid molecules encoding any of the HCVR, LCVR, and/or CDR sequences as set forth in Table 1. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced.

In another aspect, the invention provides a pharmaceutical composition comprising a recombinant human antibody or fragment thereof which specifically binds LEPR and a pharmaceutically acceptable carrier. In a related aspect, the invention features a composition which is a combination of an anti-LEPR antibody and a second therapeutic agent. In one embodiment, the second therapeutic agent is any agent that is advantageously combined with an anti-LEPR antibody.

In yet another aspect, the invention provides therapeutic methods for enhancing or stimulating LEPR signaling using an anti-LEPR antibody or antigen-binding portion of an antibody of the invention. The therapeutic methods according to this aspect of the invention comprise administering a therapeutically effective amount of a pharmaceutical composition comprising an antibody or antigen-binding fragment of an antibody of the invention to a subject in need thereof. The disorder treated is any disease or condition which is improved, ameliorated, inhibited or prevented by stimulating or activating LEPR signaling, or otherwise mimicking the natural activity of leptin in vitro or in vivo.

Other embodiments will become apparent from a review of the ensuing detailed description.

### BRIEF DESCRIPTION OF THE FIGURES

FIG. 1 depicts the binding of dimeric human LEPR to human Leptin in the presence of increasing concentrations of test anti-LEPR antibodies or control molecules, as measured by ELISA (absorbance at 450 nm).

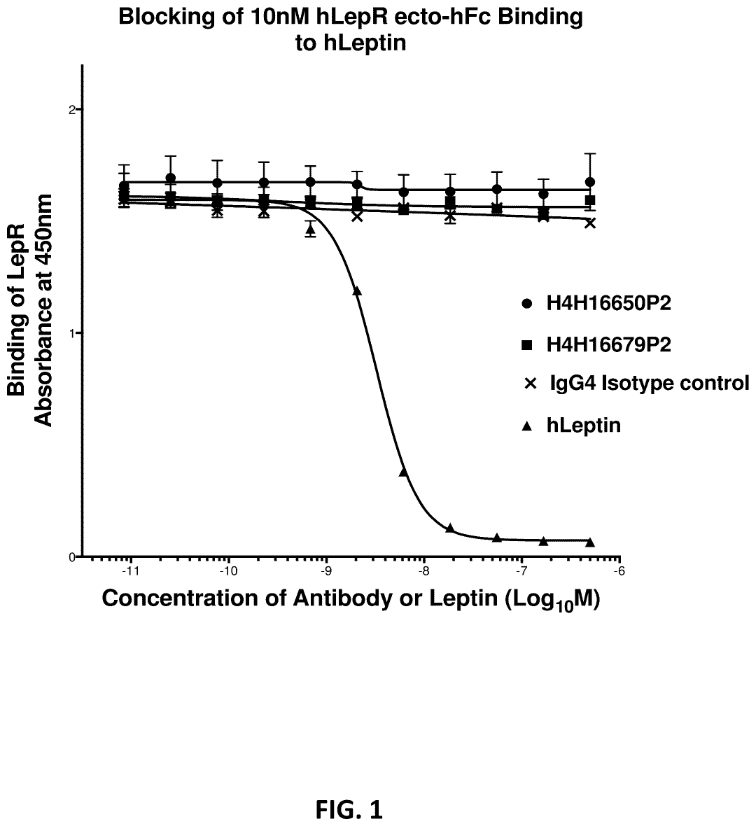

FIGS. 2A-2C illustrates the extent of LEPR signaling in HEK293 cells expressing either wild-type LEPR (circles), a signaling-defective LEPR mutant (A409E, squares), or a signaling-impaired LEPR mutant (P316T, triangles). LEPR signaling is expressed as ratio of pSTAT3-Y705/STAT3, measured by densitometry from Western blots prepared from cells treated with increasing concentrations of leptin (FIG. 2A), H4H16650 (FIG. 2B), or H4H16679 (FIG. 2C).

FIG. 3 shows the average daily food intake of leptin-deficient mice dosed with either an isotype control antibody at 3 mg/kg, or a LEPR antibody selected from H4H16650P2, H4H16679P2, H4H17319P2 or H4H17321P2 at 3 mg/kg.

FIG. 4 shows the average percent change in body weight of mice dosed with either an isotype control antibody at 3 mg/kg, or a LEPR antibody selected from H4H16650P2, H4H16679P2, H4H17319P2 or H4H17321P2 at 3 mg/kg.

FIG. 5 shows the average fat mass for animals in each antibody treatment group quantified by μCT 1 day prior to (bars not shaded) and 6 days following antibody treatment (shaded bars) expressed as mean±SEM.

FIG. 6 shows the percent change in body weight of mice fed 30 mg/kg of an antibody selected from H4H18482P2, H4H18487P2, H4H18492P2 or an isotype control.

FIGS. 7A-7B. FIG. 7A shows the fat mass of mice before dosing with anti-LEPR antibodies H4H18482P2, H4H18487P2 or H4H18492P2. FIG. 7B shows the fat mass of mice treated with 30 mg/kg of H4H18482P2, H4H18487P2 or H4H18492P2.

FIG. 8 shows that anti-LEPR antibodies tested activated monkey (Mf) LEPR in an IMR-32/STAT3-luc/Mf LEPR cell line.

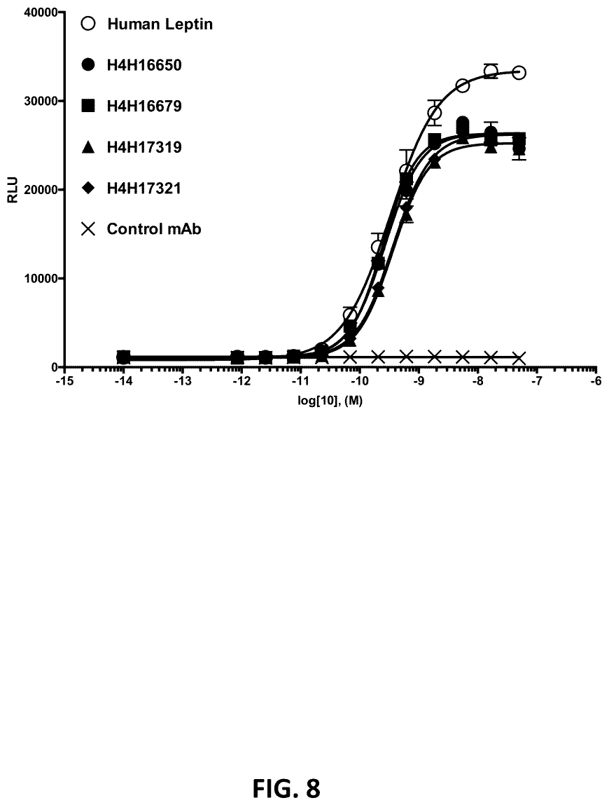

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

The expression “leptin receptor,” “LEPR,” and the like, as used herein, refers to the human leptin receptor, comprising the amino acid sequence as set forth in SEQ ID NO:113 (see also UniProtKB/Swiss-Prot Accession No. P48357). Alternative names for LEPR used in the scientific literature include “OB receptor,” “OB-R,” and “CD295.” LEPR is also referred to as “WSX” (see, e.g., U.S. Pat. No. 7,524,937). The expression “LEPR” includes both monomeric and multimeric (e.g., dimeric) LEPR molecules. As used herein, the expression “monomeric human LEPR” means a LEPR protein or portion thereof that does not contain or possess any multimerizing domains and that exists under normal conditions as a single LEPR molecule without a direct physical connection to another LEPR molecule. An exemplary monomeric LEPR molecule is the molecule referred to herein as “hLEPR.mmh” comprising the amino acid sequence of SEQ ID NO:114 (see, e.g., Example 3, herein). As used herein, the expression “dimeric human LEPR” means a construct comprising two LEPR molecules connected to one another through a linker, covalent bond, non-covalent bond, or through a multimerizing domain such as an antibody Fc domain. An exemplary dimeric LEPR molecule is the molecule referred to herein as “hLEPR.mFc” comprising the amino acid sequence of SEQ ID NO:115 (see, e.g., Example 3, herein), or the molecule referred to herein as “hLEPR.hFc” comprising the amino acid sequence of SEQ ID NO:116. As used herein, expressions such “anti-LEPR antibody,” “antibody that specifically binds LEPR,” “LEPR-specific binding protein,” and the like, unless specifically indicated otherwise, refer to molecules that bind full length human LEPR, monomeric human LEPR, dimeric human LEPR, or other constructs that comprise or consist of the LEPR extracellular domain.

All references to proteins, polypeptides and protein fragments herein are intended to refer to the human version of the respective protein, polypeptide or protein fragment unless explicitly specified as being from a non-human species. Thus, the expression “LEPR” means human LEPR unless specified as being from a non-human species, e.g., “mouse LEPR,” “monkey LEPR,” etc.

As used herein, the expression “cell surface-expressed LEPR” means one or more LEPR protein(s), or the extracellular domain thereof, that is/are expressed on the surface of a cell in vitro or in vivo, such that at least a portion of a LEPR protein is exposed to the extracellular side of the cell membrane and is accessible to an antigen-binding portion of an antibody. A “cell surface-expressed LEPR” can comprise or consist of a LEPR protein expressed on the surface of a cell which normally (e.g., in the native or wild-type state) expresses LEPR protein. Alternatively, “cell surface-expressed LEPR” can comprise or consist of LEPR protein expressed on the surface of a cell that normally does not express human LEPR on its surface but has been artificially engineered to express LEPR on its surface.

As used herein, the expressions such as “anti-LEPR antibody,” or “antibody that binds human leptin receptor,” include both monovalent antibodies with a single specificity, as well as bispecific antibodies comprising a first arm that binds LEPR and a second arm that binds a second (target) antigen, wherein the anti-LEPR arm comprises any of the HCVR/LCVR or CDR sequences as set forth in Table 1 herein.

The term “antibody”, as used herein, means any antigen-binding molecule or molecular complex comprising at least one complementarity determining region (CDR) that specifically binds to or interacts with a particular antigen (e.g., LEPR). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-LEPR antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

In certain embodiments of the invention, the anti-LEPR antibodies of the invention are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The present invention encompasses antibodies having one or more mutations in the hinge, C_{H}2 or C_{H}3 region which may be desirable, for example, in production, to improve the yield of the desired antibody form.

The antibodies of the invention may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The present invention includes variants of the anti-LEPR antibodies disclosed herein comprising one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention includes anti-LEPR antibodies and antigen-binding fragments thereof that comprise amino acid sequences that are substantially similar or substantially identical to one or more variable domain or CDR amino acid sequences as found in any of the exemplary anti-LEPR antibodies disclosed herein.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Anti-LEPR Antibodies Comprising Fc Variants

According to certain embodiments of the present invention, anti-LEPR antibodies are provided comprising an Fc domain comprising one or more mutations which enhance or diminish antibody binding to the FcRn receptor, e.g., at acidic pH as compared to neutral pH. For example, the present invention includes anti-LEPR antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present invention includes anti-LEPR antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present invention.

The anti-LEPR antibodies of the present invention may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments of the present invention, the modified Fc domain is a variant IgG1 Fc or a variant IgG4 Fc comprising a substitution in the hinge region. For example, a modified Fc for use in the context of the present invention may comprise a variant IgG1 Fc wherein at least one amino acid of the IgG1 Fc hinge region is replaced with the corresponding amino acid from the IgG2 Fc hinge region. Alternatively, a modified Fc for use in the context of the present invention may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present invention are set forth in US Patent Application Publication No. 2014/0243504, the disclosure of which is hereby incorporated by reference in its entirety, as well as any functionally equivalent variants of the modified Fc regions set forth therein.

Other modified Fc domains and Fc modifications that can be used in the context of the present invention include any of the modifications as set forth in US 2014/0171623; U.S. Pat. No. 8,697,396; US 2014/0134162; WO 2014/043361, the disclosures of which are hereby incorporated by reference in their entireties. Methods of constructing antibodies or other antigen-binding fusion proteins comprising a modified Fc domain as described herein are known in the art.

### Biological Characteristics of the Antibodies

The present invention includes antibodies and antigen-binding fragments thereof that bind human LEPR and activate LEPR signaling. Such antibodies may be referred to herein as “agonist antibodies.” In the context of the present invention, “activation of LEPR signaling” means the stimulation of an intracellular effect that normally results from the interaction of leptin with LEPR in cells that express LEPR. In certain embodiments, “activation of LEPR signaling” means the transcriptional activation of STAT3, which can be detected using any method that can measure or identify, directly or indirectly, STAT3 activity, e.g., using a labeled version of STAT3 expressed in a reporter cell line. For example, the present invention includes antibodies and antigen-binding fragments thereof that activate LEPR signaling in a cell-based reporter assay, e.g., using a cell based assay format as defined in Example 7 herein, or a substantially similar assay. Cell-based reporter assays that detect LEPR activation, such as the assay set forth in Example 7 herein, can produce a detectable signal that may be expressed in terms of an EC₅₀ value (i.e., the antibody concentration required to produce half-maximal signaling) and/or a percentage of the maximal signaling observed in the presence of leptin. In certain exemplary embodiments of the present invention, anti-LEPR antibodies are provided that activate LEPR signaling with an EC₅₀ value of less than about 12.0 nM in a cell-based reporter assay, e.g., using an assay format as defined in Example 7 herein, or a substantially similar assay. In certain exemplary embodiments of the present invention, anti-LEPR antibodies are provided that activate LEPR signaling with maximum percent activation relative to leptin signaling of greater than about 65% in a cell-based reporter assay, e.g., using an assay format as defined in Example 7 herein, or a substantially similar assay.

The present invention includes antibodies and antigen-binding fragments thereof that bind monomeric human LEPR with high affinity. For example, the present invention includes anti-LEPR antibodies that bind monomeric human LEPR (e.g., hLEPR.mmh, SEQ ID NO:114) with a K_{D} of less than about 150 nM as measured by surface plasmon resonance at 25° C. or 37° C., e.g., using an assay format as defined in Example 3 herein, or a substantially similar assay. According to certain embodiments, anti-LEPR antibodies are provided that bind monomeric human LEPR at 25° C. with a K_{D} of less than about 150 nM, less than about 140 nM, less than about 130 nM, less than about 120 nM, less than about 110 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 9 nM, less than about 8 nM, less than about 7 nM, less than about 6 nM, less than about 5 nM, less than about 4 nM, less than about 3 nM, less than about 2 nM, less than about 1 nM, less than about 900 pM, less than about 800 pM, less than about 700 pM, less than about 600 pM, less than about 500 pM, less than about 400 pM, or less than about 300 pM, as measured by surface plasmon resonance, e.g., using an assay format as defined in Example 3 herein, or a substantially similar assay.

The present invention also includes antibodies and antigen-binding fragments thereof that bind monomeric human LEPR (e.g., hLEPR.mmh, SEQ ID NO:114) with a dissociative half-life (t½) of greater than about 50 minutes as measured by surface plasmon resonance at 25° C. or 37° C., e.g., using an assay format as defined in Example 3 herein, or a substantially similar assay. According to certain embodiments, anti-LEPR antibodies are provided that bind monomeric human LEPR at 25° C. with a t½ of greater than about 50 minutes, greater than about 55 minutes, greater than about 60 minutes, greater than about 65 minutes, or longer, as measured by surface plasmon resonance, e.g., using an assay format as defined in Example 3 herein, or a substantially similar assay.

The present invention also includes antibodies and antigen-binding fragments thereof that bind dimeric human LEPR (e.g., hLEPR.mFc, SEQ ID NO:115) with high affinity. For example, the present invention includes anti-LEPR antibodies that bind dimeric human LEPR with a K_{D} of less than about 1.5 nM as measured by surface plasmon resonance at 25° C. or 37° C., e.g., using an assay format as defined in Example 3 herein, or a substantially similar assay. According to certain embodiments, anti-LEPR antibodies are provided that bind dimeric human LEPR at 25° C. with a K_{D} of less than about 150 nM, less than about 130 nM, less than about 110 nM, less than about 80 nM, less than about 70 nM, less than about 60 nM, less than about 50 nM, less than about 40 nM, less than about 30 nM, less than about 20 nM, or less than about 10 nM, as measured by surface plasmon resonance, e.g., using an assay format as defined in Example 3 herein, or a substantially similar assay.

The present invention also includes antibodies and antigen-binding fragments thereof that bind dimeric human LEPR (e.g., hLEPR.mFc, SEQ ID NO:115) with a dissociative half-life (t½) of greater than about 10 minutes as measured by surface plasmon resonance at 25° C. or 37° C., e.g., using an assay format as defined in Example 3 herein, or a substantially similar assay. According to certain embodiments, anti-LEPR antibodies are provided that bind dimeric human LEPR at 25° C. with a t½ of greater than about 10, greater than about 15 minutes, greater than about 20 minutes, greater than about 25 minutes, greater than about 30 minutes, greater than about 40 minutes, greater than about 50 minutes, greater than about 60 minutes, greater than about 70 minutes, or longer, as measured by surface plasmon resonance, e.g., using an assay format as defined in Example 3 herein, or a substantially similar assay.

The present invention also includes antibodies and antigen-binding fragments thereof that bind LEPR in complex with human leptin (“LEPR in complex with human leptin” may also be represented by the expression “leptin:LEPR”). For example, the present invention includes antibodies and antigen-binding fragments thereof that are capable of binding to a pre-formed complex comprising hLEPR and human leptin. That is, according to certain embodiments, the interaction between anti-LEPR antibodies and LEPR is not inhibited by the presence of leptin in complex with LEPR; likewise, the interaction between leptin and LEPR, according to this aspect of the invention, is not inhibited by the presence of an anti-LEPR antibody. An exemplary assay format for determining whether an antibody or antigen-binding fragment thereof binds to LEPR in complex with human leptin is set forth in Example 4 herein.

Similarly, the present invention also includes antibodies and antigen-binding fragments thereof that bind LEPR and do not block the LEPR:leptin interaction. For example, the present invention includes antibodies and antigen-binding fragments thereof that are capable of binding LEPR, thereby producing an antibody:LEPR complex, wherein the resulting antibody:LEPR complex is capable of interacting with leptin to produce a three-member complex comprising the antibody, LEPR and leptin. An exemplary assay format for determining whether an antibody or antigen-binding fragment thereof is capable of binding LEPR in a manner that does not block or interfere with the interaction between LEPR and leptin is set forth in Example 5 herein.

The present invention also includes antibodies and antigen-binding fragments thereof that bind cell surface-expressed LEPR in the presence and/or absence of human leptin. Cell surface-expressed LEPR means LEPR or a portion thereof (e.g., an extracellular portion of LEPR) expressed on the surface of a cell, either naturally or in an engineered cell line, such that an antibody or antigen-binding fragment thereof is capable of binding to the LEPR molecule. In certain embodiments, cell surface-expressed LEPR includes recombinant complexes comprising an extracellular domain of LEPR connected to a cell via a tag or anchor (e.g., a GPI anchor as illustrated in Example 6 herein). According to this aspect of the invention, antibodies are provided which are capable of binding cell surface-expressed LEPR in the absence of leptin, and are also capable of binding cell surface-expressed LEPR in the presence of leptin (i.e., under circumstances wherein leptin is capable of binding to cell surface-expressed leptin). That is, according to certain embodiments, the interaction between anti-LEPR antibodies and cell surface-expressed LEPR is not inhibited by the presence of leptin in complex with cell surface-expressed LEPR. Antibodies according to this aspect of the invention are capable of forming a three-member complex on the surface of a cell comprising the antibody, cell surface-expressed LEPR and leptin. An exemplary assay format for determining whether an antibody or antigen-binding fragment thereof is capable of binding cell surface-expressed LEPR in the presence and absence of human leptin is set forth in Example 6 herein.

The antibodies of the present invention may possess one or more of the aforementioned biological characteristics, or any combination thereof. The foregoing list of biological characteristics of the antibodies of the invention is not intended to be exhaustive. Other biological characteristics of the antibodies of the present invention will be evident to a person of ordinary skill in the art from a review of the present disclosure including the working Examples herein.

### Epitope Mapping and Related Technologies

The present invention also includes anti-LEPR antibodies comprising variants of any of the HCVR, LCVR, and/or CDR amino acid sequences disclosed herein having one or more conservative substitutions. For example, the present invention includes anti-LEPR antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein. In certain embodiments, the present invention provides anti-LEPR antibodies comprising variant HCVR, LCVR and/or CDR amino acid sequences relative to the sequences set forth in Table 1 herein (e.g., comprising conservative amino acid substitutions), wherein such variant antibodies nonetheless exhibit one or more functions and/or properties of the exemplary anti-LEPR antibodies disclosed herein.

The extracellular domain of human LEPR contains an N-terminal cytokine receptor homology domain (CRH-1), an immunoglobulin-like (Ig) domain, and a second CRH domain (CRH-2) that is referred to as the leptin-binding domain (LBD). (Carpenter et al. (2012) Structure 20:487-97). Furthermore, LEPR shares the greatest homology and similar extracellular domain size and organization with granulocyte colony stimulating factor (GCSF) and glycoprotein 130 (gp13). (Haniu et al. (1998) J Biol Chem 273 (44): 28691-699).

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The present invention includes anti-LEPR antibodies that interact with one or more epitopes found within amino acids M1-D839 of human LEPR (SEQ ID NO: 113). As set forth in Example 11, 201 peptides from human LEPR had significantly reduced deuteration uptake when bound to antibody H4H16650P2. The peptides corresponding to amino acids 162-169 (amino acids LYVLPEVL of human LEPR, SEQ ID NO: 113) and 170-191 (amino acids EDSPLVPQKGSF of human LEPR, SEQ ID NO: 113) had slower deuteration rates when bound to H4H16650P2, indicating that this antibody binds at least two human LEPR epitopes having the sequences LYVLPEVL or EDSPLVPQKGSF (amino acids 162-169 or 170-191, respectively of SEQ ID NO: 113).

The epitope to which the antibodies of the present invention bind may consist of a single contiguous sequence of 3 or more (e.g., 3, 4, 5, 6, 7, 8, 9, 10, 11, 12, 13, 14, 15, 16, 17, 18, 19, 20 or more) amino acids of a LEPR protein. Alternatively, the epitope may consist of a plurality of non-contiguous amino acids (or amino acid sequences) of LEPR. In some embodiments, the epitope is located on or near the leptin-binding domain of LEPR. In other embodiments, the epitope is located at a region distinct from the leptin-binding domain of LEPR, e.g., at a location on the surface of LEPR at which an antibody, when bound to such an epitope, does not interfere with leptin binding to LEPR.

Various techniques known to persons of ordinary skill in the art can be used to identify the amino acids within an epitope recognized by a particular antibody. Exemplary techniques include, e.g., alanine scanning mutational analysis, peptide blot analysis, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267 (2): 252-259; Engen and Smith (2001) Anal. Chem. 73: 256A-265A. X-ray crystallography analysis of an antibody in complex with its antigen may also be used to identify the amino acids within a polypeptide with which an antibody interacts.

The present invention further includes anti-LEPR antibodies that bind to the same epitope as any of the specific exemplary antibodies described herein (e.g. antibodies comprising any of the amino acid sequences as set forth in Table 1 herein). Likewise, the present invention also includes anti-LEPR antibodies that compete for binding to LEPR with any of the specific exemplary antibodies described herein (e.g. antibodies comprising any of the amino acid sequences as set forth in Table 1 herein).

One can determine whether an antibody binds to the same epitope as, or competes for binding with, a reference anti-LEPR antibody by using routine methods known in the art and exemplified herein. For example, to determine if a test antibody binds to the same epitope as a reference anti-LEPR antibody of the invention, the reference antibody is allowed to bind to a LEPR protein. Next, the ability of a test antibody to bind to the LEPR molecule is assessed. If the test antibody is able to bind to LEPR following saturation binding with the reference anti-LEPR antibody, it can be concluded that the test antibody binds to a different epitope than the reference anti-LEPR antibody. On the other hand, if the test antibody is not able to bind to the LEPR molecule following saturation binding with the reference anti-LEPR antibody, then the test antibody may bind to the same epitope as the epitope bound by the reference anti-LEPR antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-LEPR antibody, the above-described binding methodology is performed in two orientations: In a first orientation, the reference antibody is allowed to bind to a LEPR protein under saturating conditions followed by assessment of binding of the test antibody to the LEPR molecule. In a second orientation, the test antibody is allowed to bind to a LEPR molecule under saturating conditions followed by assessment of binding of the reference antibody to the LEPR molecule. If, in both orientations, only the first (saturating) antibody is capable of binding to the LEPR molecule, then it is concluded that the test antibody and the reference antibody compete for binding to LEPR. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Preparation of Human Antibodies

The anti-LEPR antibodies of the present invention can be fully human antibodies. Methods for generating monoclonal antibodies, including fully human monoclonal antibodies are known in the art. Any such known methods can be used in the context of the present invention to make human antibodies that specifically bind to human LEPR.

Using VELOCIMMUNE™ technology, for example, or any other similar known method for generating fully human monoclonal antibodies, high affinity chimeric antibodies to LEPR are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-LEPR antibody. While the constant region selected may vary according to specific use, high affinity antigen-binding and target specificity characteristics reside in the variable region. In certain instances, fully human anti-LEPR antibodies are isolated directly from antigen-positive B cells.

### Bioequivalents

The anti-LEPR antibodies and antibody fragments of the present invention encompass proteins having amino acid sequences that vary from those of the described antibodies but that retain the ability to bind human LEPR. Such variant antibodies and antibody fragments comprise one or more additions, deletions, or substitutions of amino acids when compared to parent sequence, but exhibit biological activity that is essentially equivalent to that of the described antibodies. Likewise, the anti-LEPR antibody-encoding DNA sequences of the present invention encompass sequences that comprise one or more additions, deletions, or substitutions of nucleotides when compared to the disclosed sequence, but that encode an anti-LEPR antibody or antibody fragment that is essentially bioequivalent to an anti-LEPR antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-LEPR antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-LEPR antibody variants comprising amino acid changes which modify the glycosylation characteristics of the antibodies, e.g., mutations which eliminate or remove glycosylation.

### Species Selectivity and Species Cross-Reactivity

The present invention, according to certain embodiments, provides anti-LEPR antibodies that bind to human LEPR but not to LEPR from other species. The present invention also includes anti-LEPR antibodies that bind to human LEPR and to LEPR from one or more non-human species. For example, the anti-LEPR antibodies of the invention may bind to human LEPR and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee LEPR. According to certain exemplary embodiments of the present invention, anti-LEPR antibodies are provided which specifically bind human LEPR and cynomolgus monkey (e.g., Macaca fascicularis) LEPR. Other anti-LEPR antibodies of the invention bind human LEPR but do not bind, or bind only weakly, to cynomolgus monkey LEPR.

### Multispecific Antibodies

The antibodies of the present invention may be monospecific or multispecific (e.g., bispecific). Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-LEPR antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity.

The present invention includes bispecific antibodies wherein one arm of an immunoglobulin binds human LEPR, and the other arm of the immunoglobulin is specific for a second antigen. The LEPR-binding arm can comprise any of the HCVR/LCVR or CDR amino acid sequences as set forth in Table 1 herein.

An exemplary bispecific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies. Variations on the bispecific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Formulation and Administration

The invention provides pharmaceutical compositions comprising the anti-LEPR antibodies or antigen-binding fragments thereof of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. In an adult patient, it may be advantageous to intravenously administer the antibody of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering anti-LEPR antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the Antibodies

The present invention includes methods comprising administering to a subject in need thereof a therapeutic composition comprising an anti-LEPR antibody (e.g., an anti-LEPR antibody comprising any of the HCVR/LCVR or CDR sequences as set forth in Table 1 herein). The therapeutic composition can comprise any of the anti-LEPR antibodies disclosed herein, or antigen-binding fragments thereof, and a pharmaceutically acceptable carrier or diluent.

The antibodies of the invention are useful, inter alia, for the treatment, prevention and/or amelioration of any disease or disorder associated with or mediated by leptin deficiency, leptin resistance, hypoleptinemia, or otherwise treatable by stimulating or activating LEPR signaling or mimicking the natural activity of leptin in vitro or in vivo. For example, the antibodies and antigen-binding fragments thereof of the present invention are useful for treating lipodystrophy conditions. Exemplary lipodystrophy conditions that are treatable by the antibodies and antigen-binding fragments of the present invention include, e.g., congenital generalized lipodystrophy, acquired generalized lipodystrophy, familial partial lipodystrophy, acquired partial lipodystrophy, centrifugal abdominal lipodystrophy, lipoatrophia annularis, localized lipodystrophy, and HIV-associated lipodystrophy.

The present invention also includes anti-LEPR antibodies and antigen-binding fragments thereof that are useful for restoring leptin signaling to cells, tissues and organs expressing one or more LEPR mutations associated with obesity. For example, certain LEPR mutants have been identified that exhibit no, or reduced signaling in the presence of leptin and are associated with obesity and related disorders. As used herein, a LEPR mutant that exhibits no signaling in the presence of leptin is referred to as a “signaling-defective LEPR mutant.” An exemplary signaling-defective LEPR mutation is LEPR-A409E (Farooqi et al., 2007, N Engl J Med 356 (3): 237-247). As used herein, a LEPR mutant that exhibits reduced signaling in the presence of leptin (as compared to wild-type LEPR) is referred to as a “signaling-impaired LEPR mutant.” An exemplary signaling-impaired LEPR mutation is LEPR-P316T (Mazen et al., 2011, Mol Genet Metab 102:461-464). Thus, the present invention includes anti-LEPR antibodies and antigen-binding fragments thereof that are useful for the treatment, prevention and/or amelioration of diseases and disorders caused by or associated with one or more signaling-defective (e.g., A409E) and/or signaling-impaired (e.g., P316T) LEPR mutants.

The anti-LEPR antibodies and antigen-binding fragments thereof of the present invention are also useful for the treatment or prevention of one or more diseases or disorders selected from the group consisting of obesity, metabolic syndrome, diet-induced food craving, functional hypothalamic amenorrhea, type 1 diabetes, type 2 diabetes, insulin resistance, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, lipodystrophies, Leprechaunism/Donohue syndrome, Rabson-Mendenhall syndrome.

In the context of the methods of treatment described herein, the anti-LEPR antibody may be administered as a monotherapy (i.e., as the only therapeutic agent) or in combination with one or more additional therapeutic agents (examples of which are described elsewhere herein).

### Combination Therapies and Formulations

The present invention includes compositions and therapeutic formulations comprising any of the anti-LEPR antibodies described herein in combination with one or more additional therapeutically active components, and methods of treatment comprising administering such combinations to subjects in need thereof.

The anti-LEPR antibodies of the present invention may be co-formulated with and/or administered in combination with one or more additional therapeutically active component(s), such as. e.g., pharmaceutical products prescribed for the treatment of obesity, hypercholesterolemia, hyperlipidemia, type 2 diabetes, type 1 diabetes, appetite control, infertility, etc. Examples of such additional therapeutically active components include, e.g., recombinant human leptin (e.g., metreleptin [MYALEPT]), PCSK9 inhibitors (e.g., anti-PCSK9 antibodies [alirocumab, evolocumab, bococizumab, lodelcizumab, ralpancizumab, etc.]), statins (atorvastatin, rosuvastatin, cerivastatin, pitavastatin, fluvastatin, simvastatin, lovastatin, pravastatin, etc.), ezetimibe, insulin, insulin variants, insulin secretagogues, metformin, sulfonylureas, sodium glucose cotransporter 2 (SGLT2) Inhibitors (e.g., dapaglifozin, canaglifozin, empagliflozin, etc.), GLP-1 agonists/analogues (e.g., extendin-4, exenatide, liraglutide, lixisenatide, albiglutide, dulaglutide, etc.), glucagon (GCG) inhibitors (e.g., anti-GCG antibodies), glucagon receptor (GCGR) inhibitors (e.g., anti-GCGR antibodies, small molecule GCGR antagonists, GCGR-specific antisense oligonucleotides, anti-GCGR aptamers [e.g., Spiegelmers], etc.), angiopoietin-like protein (ANGPTL) inhibitors (e.g., anti-ANGPTL3 antibodies, anti-ANGPTL4 antibodies, anti-ANGPTL8 antibodies, etc.), Phentermine, Orlistat, Topiramate, Bupropion, Topiramate/Phentermine, Bupropion/Naltrexone, Bupropion/Zonisamide, Pramlintide/Metrelepin, Lorcaserin, Cetilistat, Tesofensine, Velneperit, etc.

The additional therapeutically active component(s), e.g., any of the agents listed above or derivatives thereof, may be administered just prior to, concurrent with, or shortly after the administration of an anti-LEPR antibody of the present invention; (for purposes of the present disclosure, such administration regimens are considered the administration of an anti-LEPR antibody “in combination with” an additional therapeutically active component). The present invention includes pharmaceutical compositions in which an anti-LEPR antibody of the present invention is co-formulated with one or more of the additional therapeutically active component(s) as described elsewhere herein.

### Administration Regimens

According to certain embodiments of the present invention, multiple doses of an anti-LEPR antibody (or a pharmaceutical composition comprising a combination of an anti-LEPR antibody and any of the additional therapeutically active agents mentioned herein) may be administered to a subject over a defined time course. The methods according to this aspect of the invention comprise sequentially administering to a subject multiple doses of an anti-LEPR antibody of the invention. As used herein, “sequentially administering” means that each dose of anti-LEPR antibody is administered to the subject at a different point in time, e.g., on different days separated by a predetermined interval (e.g., hours, days, weeks or months). The present invention includes methods which comprise sequentially administering to the patient a single initial dose of an anti-LEPR antibody, followed by one or more secondary doses of the anti-LEPR antibody, and optionally followed by one or more tertiary doses of the anti-LEPR antibody.

The terms “initial dose,” “secondary doses,” and “tertiary doses,” refer to the temporal sequence of administration of the anti-LEPR antibody of the invention. Thus, the “initial dose” is the dose which is administered at the beginning of the treatment regimen (also referred to as the “baseline dose,” “loading dose,” “starting dose,” and the like); the “secondary doses” are the doses which are administered after the initial dose; and the “tertiary doses” are the doses which are administered after the secondary doses. The initial, secondary, and tertiary doses may all contain the same amount of anti-LEPR antibody, but generally may differ from one another in terms of frequency of administration. In certain embodiments, however, the amount of anti-LEPR antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

### Diagnostic and Analytic Uses of the Antibodies

The anti-LEPR antibodies of the present invention may also be used to detect and/or measure LEPR, or LEPR-expressing cells in a sample, e.g., for diagnostic purposes. For example, an anti-LEPR antibody, or fragment thereof, may be used to diagnose a condition or disease characterized by aberrant expression (e.g., over-expression, under-expression, lack of expression, etc.) of LEPR. Exemplary diagnostic assays for LEPR may comprise, e.g., contacting a sample, obtained from a patient, with an anti-LEPR antibody of the invention, wherein the anti-LEPR antibody is labeled with a detectable label or reporter molecule. Alternatively, an unlabeled anti-LEPR antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure LEPR in a sample include enzyme-linked immunosorbent assay (ELISA), radioimmunoassay (RIA), fluorescence-activated cell sorting (FACS), and positron emission tomography (PET) scanning.

Samples that can be used in LEPR diagnostic assays according to the present invention include any tissue or fluid sample obtainable from a patient which contains detectable quantities of LEPR protein, or fragments thereof, under normal or pathological conditions. Generally, levels of LEPR in a particular sample obtained from a healthy patient (e.g., a patient not afflicted with a disease or condition associated with abnormal LEPR levels or activity) will be measured to initially establish a baseline, or standard, level of LEPR. This baseline level of LEPR can then be compared against the levels of LEPR measured in samples obtained from individuals suspected of having a LEPR related disease or condition.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Antigen-Binding Proteins that Specifically Bind the Leptin Receptor (LEPR)

Anti-LEPR antibodies were obtained by immunizing a VELOCIMMUNE® mouse (i.e., an engineered mouse comprising DNA encoding human immunoglobulin heavy and kappa light chain variable regions) with an immunogen comprising the extracellular domain of LEPR. The antibody immune response was monitored by a LEPR-specific immunoassay. Using previously described techniques, fully human anti-LEPR antibodies were isolated and purified.

Certain biological properties of the exemplary anti-LEPR antibodies generated in accordance with the methods of this Example are described in detail in the Examples set forth below.

### Example 2. Heavy and Light Chain Variable Region Amino Acid and Nucleic Acid Sequences

Table 1 sets forth the amino acid sequence identifiers of the heavy and light chain variable regions and CDRs of selected anti-LEPR antibodies of the invention. The corresponding nucleic acid sequence identifiers are set forth in Table 2.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H16650P2 | 2 | 4 | 6 | 8 | 10 | 12 | 14 | 16 |
| H4H16679P2 | 18 | 20 | 22 | 24 | 10 | 12 | 14 | 16 |
| H4H17319P2 | 26 | 28 | 30 | 32 | 10 | 12 | 14 | 16 |
| H4H17321P2 | 34 | 36 | 38 | 40 | 10 | 12 | 14 | 16 |
| H4H18417P2 | 42 | 44 | 46 | 48 | 10 | 12 | 14 | 16 |
| H4H18438P2 | 50 | 52 | 54 | 56 | 10 | 12 | 14 | 16 |
| H4H18445P2 | 58 | 60 | 62 | 64 | 10 | 12 | 14 | 16 |
| H4H18446P2 | 66 | 68 | 70 | 72 | 10 | 12 | 14 | 16 |
| H4H18449P2 | 74 | 76 | 78 | 80 | 10 | 12 | 14 | 16 |
| H4H18482P2 | 82 | 84 | 86 | 88 | 90 | 92 | 94 | 96 |
| H4H18487P2 | 98 | 100 | 102 | 104 | 90 | 92 | 94 | 96 |
| H4H18492P2 | 106 | 108 | 110 | 112 | 90 | 92 | 94 | 96 |

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H16650P2 | 1 | 3 | 5 | 7 | 9 | 11 | 13 | 15 |
| H4H16679P2 | 17 | 19 | 21 | 23 | 9 | 11 | 13 | 15 |
| H4H17319P2 | 25 | 27 | 29 | 31 | 9 | 11 | 13 | 15 |
| H4H17321P2 | 33 | 35 | 37 | 39 | 9 | 11 | 13 | 15 |
| H4H18417P2 | 41 | 43 | 45 | 47 | 9 | 11 | 13 | 15 |
| H4H18438P2 | 49 | 51 | 53 | 55 | 9 | 11 | 13 | 15 |
| H4H18445P2 | 57 | 59 | 61 | 63 | 9 | 11 | 13 | 15 |
| H4H18446P2 | 65 | 67 | 69 | 71 | 9 | 11 | 13 | 15 |
| H4H18449P2 | 73 | 75 | 77 | 79 | 9 | 11 | 13 | 15 |
| H4H18482P2 | 81 | 83 | 85 | 87 | 89 | 91 | 93 | 95 |
| H4H18487P2 | 97 | 99 | 101 | 103 | 89 | 91 | 93 | 95 |
| H4H18492P2 | 105 | 107 | 109 | 111 | 89 | 91 | 93 | 95 |

Antibodies are typically referred to herein according to the following nomenclature: Fc prefix (e.g. “H4H,” “H1M,” “H2M,” etc.), followed by a numerical identifier (e.g. “16650,” “16679,” etc.), followed by a “P” or “N” suffix. Thus, according to this nomenclature, an antibody may be referred to herein as, e.g., “H4H16650P2,” “H4H16679P2,” etc. The Fc prefixes on the antibody designations used herein (H4H, H1M and H2M) indicate the particular Fc region isotype of the antibody. For example, an “H4H” antibody has a human IgG4 Fc, whereas an “H1M” antibody has a mouse IgG1 Fc, (all variable regions are fully human as denoted by the first ‘H’ in the antibody designation). As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Tables 1 and 2-will remain the same, and the binding properties are expected to be identical or substantially similar regardless of the nature of the Fc domain.

“Comparator mAb” as used in Examples herein refers to Fab9F8 described in Fazeli et al. (2006) J Immunol Methods 312:190-200 and Carpenter et al. (2012) Structure 20 (3): 487-97.

### Example 3: Surface Plasmon Resonance Derived Binding Affinities and Kinetic Constants of Human Monoclonal Anti-LEPR Antibodies

Equilibrium dissociation constants (K_{D} values) for LEPR binding to purified anti-LEPR monoclonal antibodies were determined using a real-time surface plasmon resonance biosensor using a Biacore 4000 instrument. All binding studies were performed in 10 mM HEPES, 150 mM NaCl, 3 mM EDTA, and 0.05% v/v Surfactant Tween-20, pH 7.4 (HBS-ET) running buffer at 25° C. and 37° C. The Biacore sensor surface was first derivatized by amine coupling with a monoclonal mouse anti-human Fc antibody (GE, #BR-1008-39) to capture anti-LEPR monoclonal antibodies. Binding studies were performed on following LEPR reagents: human LEPR extracellular domain expressed with a C-terminal myc-myc-hexahistidine tag (hLEPR.mmh; SEQ ID NO: 114), Macaca fascicularis LEPR extracellular domain expressed with a C-terminal myc-myc-hexahistidine tag (mfLEPR.mmh; SEQ ID NO: 117), human LEPR extracellular domain expressed with a C-terminal mouse IgG2a Fc tag (hLEPR.mFc; SEQ ID NO: 115), mouse LEPR extracellular domain expressed with a C-terminal myc-myc-hexahistidine tag (mLEPR.mmh; SEQ ID NO: 118) and rat LEPR extracellular domain expressed with a C-terminal myc-myc-hexahistidine tag (rLEPR.mmh; SEQ ID NO: 119). Different concentrations of LEPR reagents were first prepared in HBS-ET running buffer (100 nM-3.7 nM; 3-fold serial dilution) and were injected over anti-human Fc captured anti-LEPR monoclonal antibody surface for 4 minutes at a flow rate of 30 μL/minute, while the dissociation of monoclonal antibody bound LEPR reagent was monitored for 10 minutes in HBS-ET running buffer. Kinetic association (kₐ) and dissociation (k_{d}) rate constants were determined by fitting the real-time binding sensorgrams to a 1:1 binding model with mass transport limitation using Scrubber 2.0c curve-fitting software. Binding dissociation equilibrium constants (K_{D}) and dissociative half-lives (t½) were calculated from the kinetic rate constants as:

$${K}_{D} ( M ) = \frac{kd}{ka} , and {t}_{1 / 2} ( min ) = \frac{ln ( 2 )}{60 * kd}$$

Binding kinetics parameters for hLEPR.mmh, mfLEPR.MMH or hLEPR.mFc, binding to different anti-LEPR monoclonal antibodies of the invention at 25° C. and 37° C. are shown in Tables 3 through 8.

TABLE 3
Binding kinetics parameters of hLEPR-MMH binding
to LEPR monoclonal antibodies at 25° C.

| | | 100 nM | | | | |
|---|---|---|---|---|---|---|
| | mAb Capture | hLEPR-MMH | kₐ | k_{d} | K_{D} | t½ |
| mAb Captured | Level (RU) | Bound (RU) | (1/Ms) | (1/s) | (M) | (min) |
| H4H16650P2 | 167 ± 0.3 | 51 | 2.81E+04 | 2.23E−04 | 7.93E−09 | 52 |
| H4H16679P2 | 192 ± 0.7 | 39 | 2.34E+04 | 2.46E−04 | 1.05E−08 | 47 |
| H4H18417P2 | 163 ± 0.4 | 28 | 6.14E+04 | 7.90E−03 | 1.29E−07 | 1.5 |
| H4H18438P2 | 166 ± 0.4 | 22 | 3.00E+04 | 2.26E−03 | 7.54E−08 | 5.1 |
| H4H18445P2 | 194 ± 1.1 | 45 | 4.42E+04 | 4.78E−03 | 1.08E−07 | 2.4 |
| H4H18446P2 | 163 ± 2.4 | 16 | 1.81E+04 | 9.51E−04 | 5.25E−08 | 12 |
| H4H18449P2 | 176 ± 1.3 | 54 | 2.91E+04 | 2.35E−04 | 8.08E−09 | 49 |
| H4H18482P2 | 163 ± 0.4 | 47 | 6.31E+04 | 6.77E−03 | 1.07E−07 | 1.7 |
| H4H18487P2 | 190 ± 1.2 | 42 | 4.73E+04 | 7.03E−03 | 1.48E−07 | 1.6 |
| H4H18492P2 | 167 ± 3.1 | 87 | 8.10E+04 | 8.98E−04 | 1.11E−08 | 13 |
| H4H17319P2 | 200 ± 0.4 | 36 | 2.61E+04 | 5.29E−04 | 2.03E−08 | 22 |
| H4H17321P2 | 221 ± 0.5 | 32 | 2.36E+04 | 1.96E−04 | 8.31E−09 | 59 |
| Isotype | 171 ± 0.4 | 4 | NB* | NB* | NB* | NB* |
| Control mAb | | | | | | |

*NB indicates that no binding was observed under the current experimental conditions.

TABLE 4
Binding kinetics parameters of hLEPR-MMH binding
to LEPR monoclonal antibodies at 37° C.

| | | 100 nM | | | | |
|---|---|---|---|---|---|---|
| | mAb Capture | hLEPR-MMH | kₐ | k_{d} | K_{D} | t½ |
| mAb Captured | Level (RU) | Bound (RU) | (1/Ms) | (1/s) | (M) | (min) |
| H4H16650P2 | 210 ± 2.5 | 77 | 4.85E+04 | 9.58E−04 | 1.98E−08 | 12 |
| H4H16679P2 | 239 ± 2 | 61 | 3.84E+04 | 8.42E−04 | 2.19E−08 | 14 |
| H4H18417P2 | 206 ± 3.2 | 22 | 7.70E+04 | 1.80E−02 | 2.33E−07 | 0.6 |
| H4H18438P2 | 206 ± 2.4 | 32 | 3.38E+04 | 5.76E−03 | 1.70E−07 | 2.0 |
| H4H18445P2 | 234 ± 2 | 38 | 5.13E+04 | 1.68E−02 | 3.26E−07 | 0.7 |
| H4H18446P2 | 188 ± 3.4 | 21 | 2.12E+04 | 2.56E−03 | 1.21E−07 | 4.5 |
| H4H18449P2 | 206 ± 2.1 | 73 | 3.94E+04 | 8.15E−04 | 2.07E−08 | 14 |
| H4H18482P2 | 188 ± 0.8 | 38 | 9.53E+04 | 1.93E−02 | 2.03E−07 | 0.6 |
| H4H18487P2 | 219 ± 1.7 | 30 | 6.51E+04 | 1.86E−02 | 2.86E−07 | 0.6 |
| H4H18492P2 | 192 ± 2.2 | 93 | 1.17E+05 | 4.18E−03 | 3.59E−08 | 2.8 |
| H4H17319P2 | 264 ± 0.3 | 44 | 3.54E+04 | 3.41E−03 | 9.63E−08 | 3.4 |
| H4H17321P2 | 290 ± 0.4 | 61 | 2.95E+04 | 4.38E−04 | 1.48E−08 | 26 |
| Isotype | 193 ± 1.5 | 6 | NB* | NB* | NB* | NB* |
| Control mAb | | | | | | |

*NB indicates that no binding was observed under the current experimental conditions.

TABLE 5
Binding kinetics parameters of mfLEPR.MMH binding
to LEPR monoclonal antibodies at 25° C.

| | | 100 nM | | | | |
|---|---|---|---|---|---|---|
| | mAb Capture | mfLEP.MMH | kₐ | k_{d} | K_{D} | t½ |
| mAb Captured | Level (RU) | Bound (RU) | (1/Ms) | (1/s) | (M) | (min) |
| H4H16650P2 | 166 ± 0.6 | 93 | 6.02E+04 | 1.37E−04 | 2.27E−09 | 84 |
| H4H16679P2 | 191 ± 0.7 | 66 | 4.37E+04 | 1.41E−04 | 3.22E−09 | 82 |
| H4H18417P2 | 162 ± 0.3 | 33 | 8.83E+04 | 1.23E−02 | 1.39E−07 | 0.9 |
| H4H18438P2 | 166 ± 0.6 | 5 | IC* | IC* | IC* | IC* |
| H4H18445P2 | 193 ± 0.6 | 58 | 5.90E+04 | 4.86E−03 | 8.24E−08 | 2.4 |
| H4H18446P2 | 163 ± 2.8 | 23 | 1.93E+04 | 1.12E−03 | 5.83E−08 | 10 |
| H4H18449P2 | 175 ± 0.5 | 6 | IC* | IC* | IC* | IC* |
| H4H18482P2 | 163 ± 0.8 | 63 | 1.01E+05 | 6.74E−03 | 6.66E−08 | 1.7 |
| H4H18487P2 | 189 ± 0.5 | 59 | 7.37E+04 | 6.79E−03 | 9.21E−08 | 1.7 |
| H4H18492P2 | 165 ± 2.4 | 52 | 1.10E+05 | 1.20E−02 | 1.10E−07 | 1.0 |
| H4H17319P2 | 213 ± 0.5 | 83 | 4.00E+04 | 4.63E−04 | 1.16E−08 | 25 |
| H4H17321P2 | 236 ± 0.4 | 75 | 3.26E+04 | 1.33E−04 | 4.07E−09 | 87 |
| Isotype | 171 ± 0.4 | 0 | NB* | NB* | NB* | NB* |
| Control mAb | | | | | | |

*NB indicates that no binding was observed under the current experimental conditions.
*IC indicates that observed binding was inclusive and was unable to fit the real time binding data under the current experimental conditions.

TABLE 6
Binding kinetics parameters of mfLEPR.MMH binding
to LEPR monoclonal antibodies at 37° C.

| | | 100 nM | | | | |
|---|---|---|---|---|---|---|
| | mAb Capture | mfLEPR.MMH | kₐ | k_{d} | K_{D} | t½ |
| mAb Captured | Level (RU) | Bound (RU) | (1/Ms) | (1/s) | (M) | (min) |
| H4H16650P2 | 204 ± 1.7 | 134 | 1.22E+05 | 7.00E−04 | 5.76E−09 | 16 |
| H4H16679P2 | 232 ± 1.1 | 104 | 6.49E+04 | 6.77E−04 | 1.04E−08 | 17 |
| H4H18417P2 | 202 ± 1.3 | 28 | 1.22E+05 | 2.63E−02 | 2.17E−07 | 0.4 |
| H4H18438P2 | 203 ± 1.3 | 7 | IC* | IC* | IC* | IC* |
| H4H18445P2 | 232 ± 0.9 | 48 | 7.17E+04 | 1.90E−02 | 2.64E−07 | 0.6 |
| H4H18446P2 | 188 ± 2.9 | 30 | 2.53E+04 | 3.54E−03 | 1.40E−07 | 3.3 |
| H4H18449P2 | 202 ± 1 | 6 | IC* | IC* | IC* | IC* |
| H4H18482P2 | 187 ± 1.2 | 52 | 1.52E+05 | 2.04E−02 | 1.34E−07 | 0.6 |
| H4H18487P2 | 216 ± 0.7 | 44 | 1.10E+05 | 1.95E−02 | 1.78E−07 | 0.6 |
| H4H18492P2 | 191 ± 1.4 | 34 | 2.34E+05 | 3.94E−02 | 1.69E−07 | 0.3 |
| H4H17319P2 | 274 ± 0.5 | 113 | 5.39E+04 | 3.24E−03 | 6.01E−08 | 3.6 |
| H4H17321P2 | 304 ± 0.7 | 143 | 4.97E+04 | 2.57E−04 | 5.18E−09 | 45 |
| Isotype | 190 ± 1 | 1 | NB* | NB* | NB* | NB* |
| Control mAb | | | | | | |

*NB indicates that no binding was observed under the current experimental conditions.
*IC indicates that observed binding was inclusive and was unable to fit the real time binding data under the current experimental conditions.

TABLE 7
Binding kinetics parameters of hLEPR.mFc binding
to LEPR monoclonal antibodies at 25° C.

| | | 100 nM | | | | |
|---|---|---|---|---|---|---|
| | mAb Capture | hLEPR-mFc | kₐ | k_{d} | K_{D} | t½ |
| mAb Captured | Level (RU) | Bound (RU) | (1/Ms) | (1/s) | (M) | (min) |
| H4H16650P2 | 165 ± 0.2 | 102 | 1.06E+05 | 8.32E−05 | 7.85E−10 | 139 |
| H4H16679P2 | 190 ± 1.2 | 78 | 5.84E+04 | 9.68E−05 | 1.66E−09 | 119 |
| H4H18417P2 | 162 ± 0.6 | 90 | 1.40E+05 | 5.63E−04 | 4.04E−09 | 21 |
| H4H18438P2 | 165 ± 1.2 | 51 | 5.19E+04 | 2.44E−04 | 4.70E−09 | 47 |
| H4H18445P2 | 192 ± 0.4 | 76 | 1.22E+05 | 4.92E−04 | 4.03E−09 | 23 |
| H4H18446P2 | 162 ± 2.8 | 20 | 3.20E+04 | 2.08E−04 | 6.48E−09 | 56 |
| H4H18449P2 | 174 ± 0.6 | 116 | 7.05E+04 | 6.82E−05 | 9.64E−10 | 169 |
| H4H18482P2 | 162 ± 0.5 | 88 | 1.44E+05 | 4.91E−04 | 3.42E−09 | 24 |
| H4H18487P2 | 188 ± 0.6 | 85 | 1.06E+05 | 6.03E−04 | 5.70E−09 | 19 |
| H4H18492P2 | 166 ± 3.2 | 129 | 2.27E+05 | 1.39E−04 | 6.13E−10 | 83 |
| H4H17319P2 | 200 ± 0.5 | 69 | 4.77E+04 | 1.64E−04 | 3.45E−09 | 70 |
| H4H17321P2 | 221 ± 0.4 | 65 | 4.10E+04 | 8.93E−05 | 2.18E−09 | 129 |
| Isotype | 170 ± 0.7 | −2 | NB* | NB* | NB* | NB* |
| Control mAb | | | | | | |

*NB indicates that no binding was observed under the current experimental conditions.

TABLE 8
Binding kinetics parameters of hLEPR.mFc binding
to LEPR monoclonal antibodies at 37° C.

| | | 100 nM | | | | |
|---|---|---|---|---|---|---|
| | mAb Capture | hLEPR-mFc | kₐ | k_{d} | K_{D} | t½ |
| mAb Captured | Level (RU) | Bound (RU) | (1/Ms) | (1/s) | (M) | (min) |
| H4H16650P2 | 199 ± 1.9 | 145 | 1.57E+05 | 2.80E−04 | 1.79E−09 | 41 |
| H4H16679P2 | 229 ± 2.3 | 116 | 1.21E+05 | 3.10E−04 | 2.56E−09 | 37 |
| H4H18417P2 | 199 ± 1.1 | 111 | 1.85E+05 | 1.05E−03 | 5.64E−09 | 11 |
| H4H18438P2 | 199 ± 0.6 | 82 | 7.02E+04 | 5.98E−04 | 8.53E−09 | 19 |
| H4H18445P2 | 229 ± 2 | 104 | 1.56E+05 | 6.08E−04 | 3.89E−09 | 19 |
| H4H18446P2 | 186 ± 2.5 | 34 | 4.27E+04 | 5.48E−04 | 1.28E−08 | 21 |
| H4H18449P2 | 198 ± 1.6 | 148 | 1.33E+05 | 1.68E−04 | 1.26E−09 | 69 |
| H4H18482P2 | 185 ± 1.3 | 109 | 1.89E+05 | 7.26E−04 | 3.84E−09 | 16 |
| H4H18487P2 | 215 ± 1.5 | 99 | 1.23E+05 | 6.06E−04 | 4.93E−09 | 19 |
| H4H18492P2 | 189 ± 1.8 | 160 | 4.33E+05 | 5.00E−04 | 1.16E−09 | 23 |
| H4H17319P2 | 262 ± 0.5 | 100 | 8.51E+04 | 6.52E−04 | 7.66E−09 | 18 |
| H4H17321P2 | 289 ± 0.4 | 110 | 5.53E+04 | 1.74E−04 | 3.15E−09 | 66 |
| Isotype | 188 ± 0.8 | 1 | NB* | NB* | NB* | NB* |
| Control mAb | | | | | | |

*NB indicates that no binding was observed under the current experimental conditions.

At 25° C., anti-LEPR monoclonal antibodies bound to hLEPR-MMH with K_{D} values ranging from 7.93 nM to 148 nM, as shown in Table 5. At 37° C., anti-LEPR monoclonal antibodies bound to hLEPR-MMH with K_{D} values ranging from 14.8 nM to 326 nM, as shown in Table 4.

Ten out of 12 anti-LEPR monoclonal antibodies of the invention bound to mfLEPR.MMH. At 25° C., anti-LEPR monoclonal antibodies bound to mfLEPR.MMH with K_{D} values ranging from 2.27 nM to 139 nM, as shown in Table 7. At 37° C., anti-LEPR monoclonal antibodies bound to mfLEPR.MMH with K_{D} values ranging from 5.18 nM to 264 nM, as shown in Table 8.

At 25° C., anti-LEPR monoclonal antibodies bound to hLEPR-mFc with K_{D} values ranging from 613 pM to 5.7 nM, as shown in Table 7. At 37° C., anti-LEPR monoclonal antibodies bound to hLEPR-mFc with K_{D} values ranging from 1.16 nM to 12.8 nM, as shown in Table 8.

None of the anti-LEPR monoclonal antibodies of the invention bound to mLEPR.MMH or rLEPR.MMH at 25° C. or at 37° C. (data not shown).

### Example 4. Anti-LEPR Antibodies of the Invention Bind LEPR in the Presence of Leptin:LEPR Binding

Blocking of anti-LEPR antibodies from binding to LEPR by human Leptin was evaluated using a real-time surface plasmon resonance biosensor on a Biacore T200 instrument. The entire study was performed in 10 mM HEPES pH 7.4, 150 mM NaCl, 3 mM EDTA, and 0.05% v/v Surfactant Tween-20 (HBS-ET running buffer) at 25° C. The Biacore CM5 sensor surface was first derivatized by amine coupling human Leptin (R&D Systems, #398-LP) using standard EDC/NHS surface chemistry. A complex of human LEPR and human Leptin, was formed by injecting 20 nM of human LEPR extracellular domain expressed with a C-terminal myc-myc-hexahistidine tag (hLEPR-MMH; SEQ ID NO: 114), over the human Leptin immobilized Biacore sensor surface at a flow rate of 10 μL/minute or 25 μL/minute for 4 minutes, to achieve a binding response of approximately 200RU. To evaluate whether antibody binding to hLEPR-MMH is blocked by human Leptin, 200 nM of anti-LEPR monoclonal antibodies were injected over the preformed hLEPR-MMH: human Leptin complex, at a flow rate of 50 μL/minute or 25 μL/minute for 4-5 minutes. All of the anti-LEPR antibodies of this invention that were tested bound to the complex of hLEPR-MMH and human Leptin (“Leptn;LEPR”) with nearly similar signal strength and the observed binding, expressed in RUs, are reported in Table 9. This result indicates that human Leptin does not block the binding of hLEPR-MMH to anti-LEPR antibodies tested.

TABLE 9
Binding of anti-LEPR monoclonal antibodies to
the pre-complex of hLEPR-MMH and human Leptin.

| | hLEPR-MMH | 200 nM mAb |
|---|---|---|
| Antibody | Bound (RU) | Bound (RU) |
| H4H16650P2 | 196 | 81 |
| H4H16679P2 | 195 | 90 |
| H4H17319P2 | 196 | 92 |

### Example 5. Human Leptin Receptor Blocking ELISA

For the ELISA, human Leptin (hLeptin; R&D Systems, #398-LP-01M) was coated at a concentration of 5 μg/mL in PBS on a 96-well microtiter plate overnight at 4° C. Nonspecific binding sites were subsequently blocked using a 0.5% (w/v) solution of BSA in PBS. A constant amount of 10 nM of extracellular domain portion of LEPR protein that was expressed with a C-terminal human Fc tag (hLEPR.hFc; SEQ ID NO: 116) was titrated with anti-LEPR antibodies, hLeptin protein, or an isotype control antibody ranging from 8.5 pM to 500 nM in serial dilution. These antibody-protein or protein-protein complexes were then incubated for 1.5 hour at room temperature (RT). Complexes were subsequently transferred to microtiter plates coated with hLeptin and incubated for 2 hours at RT, the wells were washed, and plate-bound hLEPR.hFc was detected with an anti-human IgG polyclonal antibody conjugated with horseradish peroxidase (Jackson ImmunoResearch Inc, #109-035-098). Samples were developed with a TMB solution (BD Biosciences, #555214; substrate A and B mixed at 1:1 ratio as per manufacturer's instructions) to produce a colorimetric reaction and then neutralized with 1M sulfuric acid before measuring absorbance at 450 nm on a Victor X5 plate reader.

Data analysis was performed using a sigmoidal dose-response model within Prism™ software (GraphPad). Percent blockade at maximum concentration of the antibody tested was calculated as an indicator of the ability of the antibodies to block the binding of 10 nM of hLEPR.hFc to human Leptin on the plate. In the calculation, binding signal of 10 nM of hLEPR.hFc without the presence of the antibody was referenced as 100% binding or 0% blocking; and the baseline signal of buffer alone without the presence of hLEPR.hFc was referenced as 0% binding or 100% blocking. The blocking data at 500 nM antibody concentration is summarized in Table 10.

As shown in Table 10, none of the anti-LEPR antibodies of the invention demonstrated >28% blocking of the binding of hLEPR.hFc to the hLeptin coated surface. However, the Comparator Antibody and the hLeptin, as the positive control, were able to block 99% of the hLEPR.hFc binding to the hLeptin coated surface. The isotype control antibody demonstrated no measurable blocking at concentrations up to 500 nM.

TABLE 10
ELISA blocking of hLEPR.hFc binding
to hLeptin by anti-LEPR antibodies

| | 500 nM Ab Blocking of |
|---|---|
| | 10 nM hLEPR.hFc |
| | Binding to hLeptin |
| Antibody | (% blockade) |
| H4H18487P2 | 5 |
| H4H18417P2 | 16 |
| H4H18482P2 | 25 |
| H4H18492P2 | −3 |
| H4H18445P2 | 28 |
| H4H18446P2 | −5 |
| H4H18449P2 | 8 |
| H4H18438P2 | 15 |
| H4H16650P2 | −7 |
| H4H16679P2 | 7 |
| H4H17319P2 | 9 |
| H4H17321P2 | 6 |

Controls

| | |
|---|---|
| Isotype control antibody | −3 |
| Human Leptin | 99 |
| Comparator Antibody | 99 |
| Mouse IgG2a Isotype | 32 |
| control | |

### Example 6. Cell Binding by FACS Analysis with HEK293/Mycx2-hLepR(Ecto)-GPI Anchored Cells

Leptin receptor, LEPR, is a single-pass transmembrane receptor of the class I cytokine receptor family (Tartaglia et al. (1997) J Biol Chem 7: 272 (10): 6093-6). LEPR can bind to Leptin, a protein predominantly expressed by adipose tissue that is involved in regulation of food intake and metabolism (Friedman et al. (2014) J Endocrinol 223 (1): T1-8).

In order to assess cell binding by anti-LEPR antibodies HEK293 stable cell lines were generated. One cell line, known hereafter as HEK293/hLEPR-GPI, stably expressed the extracellular domain of human LEPR (amino acids 22-839 of accession #P48357 (SEQ ID NO: 113), Isoform B) with an N-terminal myc-myc tag and C-terminal peptide sequence from human carboxypeptidase M that guides the addition of GPI (Glycosylphosphatidylinositol) (Deddish et al. (1990) J. Biological Chemistry 265:25:15083-89) such that the protein can be GPI-anchored to the membrane. Another HEK293 cell line was generated to stably express the full length human LEPR (amino acids 1-1165 of accession #P48357 (SEQ ID NO:113), Isoform B) along with a luciferase reporter (Stat3-luciferase, Stat3-luc, SA Bioscience, #CLS-6028L), and is known hereafter as HEK293/Stat3-luc/hLEPR-FL. HEK293 cells with the Stat3-luciferase reporter only (HEK293/Stat3-luc) were also generated as a control cell line.

For the FACS analysis, HEK293 parental cells and HEK293/hLEPR-GPI cells were dissociated and plated onto 96-well v-bottom plates at 5×10⁵ cells/well in PBS containing 2% FBS (FACS buffer). In order to test whether the ability of anti-hLEPR antibodies to bind to cells is affected by the presence of Leptin, FACS buffer with or without 1 μM human Leptin (R&D Systems, #398-LP) was incubated with the cells for 30 minutes at 4° C., followed by the addition of anti-LEPR antibodies or control antibodies at 10 nM in FACS buffer. The cells were subsequently incubated for 30 minutes at 4° C., followed by washing and then incubation with 16 μg/mL of Alexa Fluor®-647 conjugated secondary antibody (Jackson ImmunoResearch Laboratories Inc., #109-547-003) for 30 minutes at 4° C. Cells were subsequently fixed using BD CytoFix™ (Becton Dickinson, #554655), filtered, and analyzed on a HyperCyt Flow Cytometer (Beckman Coulter). Unstained and secondary antibody alone controls were also tested for all cell lines. The results were analyzed using ForeCyt (IntelliCyt) and FlowJo version 10 software to determine the geometric means of fluorescence for viable cells. The geometric mean of fluorescence for each sample was then normalized to the geometric mean of unstained cells to obtain relative binding per condition referred to as “binding ratios”, and these binding ratios were recorded for each antibody tested.

As shown in Table 11, 9 anti-LEPR antibodies of the invention tested at 10 nM demonstrated binding to HEK293/hLEPR-GPI cells with binding ratios ranging from 824 to 3374 fold without Leptin. The anti-LEPR antibodies also bound in the presence of 1 μM Leptin with binding ratios of 398 and 4184 fold. As shown in Table 11, he Comparator Antibody tested at 10 nM demonstrated binding to HEK293/hLEPR-GPI cells with a binding ratio of 2349-fold without Leptin but showed significantly less binding to cells in the presence of 1 μM Leptin with binding ratio of 112. The anti-LEPR antibodies did not demonstrate any significant binding to the HEK293 parental cells with binding ratios with and without 1 μM Leptin ranging from 1 to 9 fold. The isotype control antibodies and secondary antibodies alone samples also did not demonstrate significant binding to either cell line with or without Leptin, with binding ratios ranging from 1 to 6 fold.

As shown in Table 12, four antibodies of the invention tested at 70 nM without Leptin, demonstrated binding to HEK293/hLEPR-GPI cells with binding ratios ranging from 707 to 1131 fold and to HEK293/Stat3-luc/hLEPR-FL cells with binding ratios ranging from 42 to 51. The anti-LEPR antibodies did not demonstrate any significant binding to the HEK293/Stat3-luc cells with binding ratios ranging from 1 to 8 fold. The isotype control antibodies and secondary antibodies alone samples also did not demonstrate significant binding to any of the cell lines tested, with binding ratios ranging from 1 to 2 fold.

TABLE 11
Binding of 10 nM anti-LEPR antibodies to HEK293/hLEPR-GPI and
HEK293 parental cells +/− 1 μM Human Leptin

| | Binding Ratio: Normalized to Unstained | | | | |
|---|---|---|---|---|---|
| | Sample of Each Cell Line | | | | |
| | No added Leptin | | 1 μM Leptin | | |
| | HEK293 | HEK293/ | HEK293 | HEK293/ | Antibody |
| Antibody | parental | hLEPR-GPI | parental | hLEPR-GPI | Type |
| H4H16650P2 | 5 | 2420 | 4 | 3124 | Agonist |
| H4H16679P2 | 5 | 2058 | 8 | 2223 | Agonist |
| H4H18417P2 | 1 | 1835 | 2 | 2604 | Potentiator |
| H4H18438P2 | 2 | 1486 | 3 | 2414 | Potentiator |
| H4H18445P2 | 2 | 2016 | 3 | 2488 | Potentiator |
| H4H18449P2 | 5 | 3374 | 9 | 3113 | Potentiator |
| H4H18482P2 | 1 | 1966 | 3 | 2704 | Potentiator |
| H4H18487P2 | 1 | 2422 | 3 | 2670 | Potentiator |
| H4H18492P2 | 3 | 2603 | 7 | 4184 | Potentiator |
| Comparitor | 6 | 2349 | 3 | 112 | N/A |
| Isotype control | 1 | 6 | 2 | 4 | N/A |
| antibody | | | | | |
| Secondary | 1 | 3 | 2 | 3 | N/A |
| antibody alone | | | | | |
| Unstained | 1 | 1 | 1 | 1 | N/A |

*Classification of antibodies as “Agonist” or “Potentiator” is based in part on the results observed in Examples 7 and 8 herein.

TABLE 12
Binding of 70 nM anti-LEPR antibodies to HEK293/hLEPR-GPI,
HEK293/Stat3-hLEPR-FL, and HEK293/Stat3-luc parental cells

| | Binding Ratio: Normalized to Unstained | | | |
|---|---|---|---|---|
| | Sample of Each Cell Line | | | |
| | | | HEK293/ | |
| | HEK293/ | HEK293/ | Stat3-luc | Antibody |
| Antibody | Stat3-luc | hLEPR-GPI | hLEPR-FL | Type |
| H4H16650P2 | 6 | 707 | 42 | Agonist |
| H4H16679P2 | 8 | 1078 | 51 | Agonist |
| H4H17319P2 | 7 | 1131 | 47 | Agonist |
| H4H17321P2 | 7 | 1126 | 46 | Agonist |
| Isotype control | 2 | 2 | 2 | |
| antibody | | | | |
| Secondary | 1 | 1 | 1 | |
| antibody alone | | | | |
| Unstained | 1 | 1 | 1 | |

### Example 7. Anti-LEPR Antibodies of the Invention Activate LEPR Signaling in the Presence or Absence of Leptin

A bioassay was developed to detect the transcriptional activation of STAT3 via LEPR activation using a reporter cell line that stably expresses full-length human LEPR (hLEPR; amino acids 1 through 1165 of accession number NP_002294.2) along with a luciferase reporter (STAT3-Luc; Qiagen, #CLS-6028L) in an IMR-32 cell line, a human neuroblastoma cell line. The resulting stable cell line, referred to as IMR-32/STAT3-Luc/hLEPR, was isolated and maintained in MEM-Earl medium supplemented with 10% FBS, NEAA, 1 ug/mL Puromycin, 100 ug/mL of Hygromycin B and Penicillin/Streptomycin/L-Glutamine (Complete Medium).

The resulting bioassay was used to measure the effect of anti-LEPR antibodies of the invention on LEPR signaling in the presence or absence of Leptin. For the bioassay, IMR-32/STAT3-Luc/hLEPR cells were plated at the density of 20,000 cells/100 ul/well for 96 well format in the complete medium, and the following day replaced with the appropriate volume of Opti-MEM medium supplemented with 1% BSA and 0.1% FBS (Assay Buffer) for 30 minutes. To measure the effect of the antibodies of the invention in the absence of Leptin, the anti-LEPR antibodies or an isotype control antibody and human Leptin (hLeptin; R&D Systems, #398-LP) were half-log serially diluted to final concentrations ranging from 100 nM to 300 fM in Assay Buffer, which were added to the cells and subsequently incubated overnight at 37° C. in 5% CO2.

To measure the effect of the antibodies of the invention in the presence of Leptin, a fixed concentration of human Leptin at 200 pM in Assay Buffer was added to the cells, immediately followed by the addition of anti-LEPR antibodies or isotype control antibody that were half-log serially diluted to final concentrations ranging from 100 nM to 300 fM. The samples were then incubated overnight at 37° C. in 5% CO2. OneGlo reagent (Promega, #E6051) was then added to the samples and luciferase activity was measured on an Envision Multilable Plate Reader (Perkin Elmer) in Luminescent mode. The relative light unit (RLU) values were obtained and the results were analyzed using nonlinear regression with GraphPad Prism software (GraphPad). The maximum RLU value obtained from the hLeptin dose response was defined as 100% activation in the IMR-32/STAT3-Luc/hLEPR assay.

As shown in Table 13, in Study 1, in the absence of hLeptin, all of the anti-LEPR antibodies tested demonstrated weak stimulation of the IMR-32/STAT3-Luc/hLEPR cells with EC₅₀ values ranging from 134 pM to 11.9 nM and maximal activation ranging from 5% to 13% respectively that of maximum activation obtained from the hLeptin dose response. In Study 2, in the absence of hLeptin, the 4 anti-LEPR antibodies tested demonstrated stimulation of the IMR-32/STAT3-Luc/hLEPR cells with EC₅₀ values ranging from 61.9 pM to 206.9 pM and maximal activation ranging from 65% to 68% respective to the maximum activation obtained from the hLeptin dose response. In Study 1, in the presence of 200 pM of hLeptin, all of the anti-LEPR antibodies tested demonstrated stimulation of the IMR-32/STAT3-Luc/hLEPR cells with EC₅₀ values ranging from 20.2 pM to 523 pM and maximal activation ranging from 66% to 107% respectively that of maximum activation obtained from the hLeptin dose response. Because these antibodies enhanced leptin-induced LEPR signaling, these antibodies were classified as “potentiators”, as defined herein. In Study 2, in the presence of 200 pM of hLeptin, the 4 anti-LEPR antibodies tested demonstrated stimulation of the IMR-32/STAT3-Luc/hLEPR cells with EC₅₀ values ranging from 51.9 pM to 257.3 pM with maximal activation ranging from 76% to 88% that of maximum activation obtained from the hLeptin dose response. LEPR signaling was not appreciably enhanced by these antibodies in the presence of leptin. The isotype control antibody did not demonstrate any measurable stimulation of the IMR-32/STAT3-Luc/hLEPR cells in any of the assays.

TABLE 13
Activation of hLEPR by anti-LEPR Antibodies

| | IMR-32/LEPR without | | IMR-32/LEPR with | |
|---|---|---|---|---|
| | human Leptin | | 200 pM human Leptin | |
| | | % | | % |
| Antibody | EC₅₀ (M) | activation | EC₅₀ (M) | activation |

Study 1

| | | | | |
|---|---|---|---|---|
| H4H18445P2 | 1.19E−08 | 5 | 4.10E−10 | 97 |
| H4H18446P2 | 3.73E−10 | 6 | 3.42E−11 | 68 |
| H4H18449P2 | 2.12E−10 | 13 | 5.23E−11 | 66 |
| H4H18438P2 | 1.49E−09 | 5 | 2.02E−11 | 76 |
| H4H18482P2 | 2.69E−10 | 7 | 1.69E−10 | 94 |
| H4H18487P2 | 8.01E−10 | 6 | 4.10E−10 | 107 |
| H4H18492P2 | 1.34E−10 | 5 | 2.74E−11 | 94 |
| H4H18417P2 | 1.53E−10 | 5 | 5.23E−10 | 87 |

Study 2

| | | | | |
|---|---|---|---|---|
| H4H16650P2 | 6.19E−11 | 68 | 5.19E−11 | 88 |
| H4H16679P2 | 8.62E−11 | 65 | 7.37E−11 | 88 |
| H4H17319P2 | 1.867E−10 | 68 | 1.914E−10 | 76 |
| H4H17321P2 | 2.069E−10 | 66 | 2.573E−10 | 76 |

### Example 8. Anti-LEPR Antibodies of the Invention Activate Signaling in Cells Expressing Signaling-Defective or Signaling-Impaired LEPR Mutants

LEPR mutants have been identified that exhibit defective or impaired leptin-mediated signaling and are associated with early-onset obesity. For example, LEPR-A409E is a signaling-defective mutant LEPR protein that does not transduce leptin signals to STAT3; the A409E mutant was originally identified as a monogenic cause of early onset obesity. (Farooqi et al., 2007, N Engl J Med 356 (3): 237-247). LEPR-P316T is a signaling-impaired mutant LEPR protein that has also been shown to be associated with early-onset obesity. (Mazen et al., 2011, Mol Genet Metab 102:461-464).

In this Example, the ability of anti-LEPR antibodies of the invention to stimulate LEPR signaling in cell lines expressing signaling-defective or signaling-impaired LEPR mutants was assessed. In particular, reporter cell lines (HEK293) were constructed expressing either wild-type LEPR, LEPR-A409E (signaling-defective) or LEPR-P316T (signaling-impaired). Cells were treated with either vehicle only, recombinant human leptin, control IgG, or agonist anti-LEPR antibodies of the present invention (H4H16650 or H4H16679), and the extent of LEPR signaling (as measured by Western blot detection of pSTAT3-Y705 expression relative to STAT3 expression) was determined.

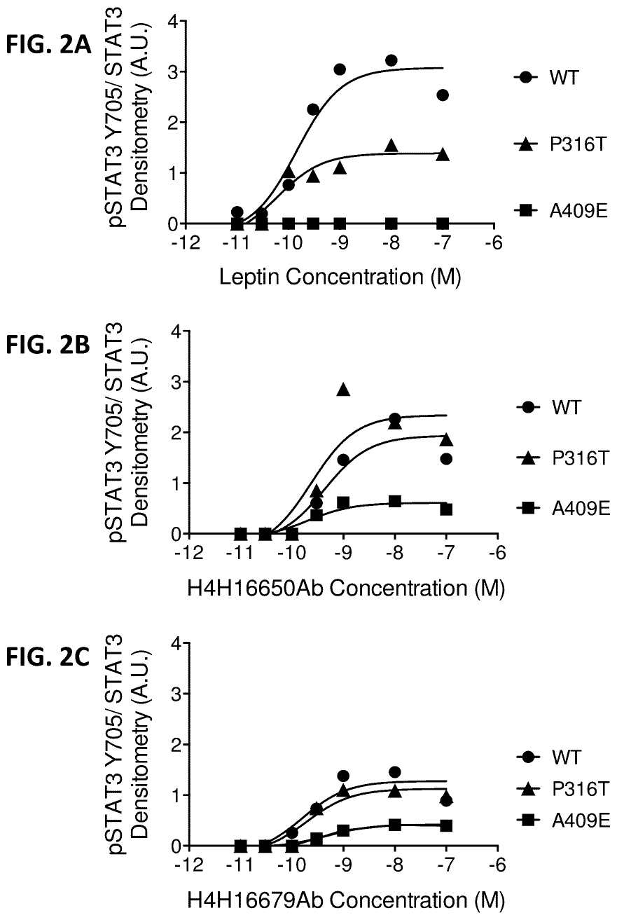

The agonist anti-LEPR antibodies of the present invention (H4H16650 and H4H16679) were shown in these experiments to stimulate LEPR signaling in cells expressing the LEPR-A409E mutant or the LEPR-P316T mutant (as measured by STAT3 expression) in a dose-dependent manner (FIG. 2, panels B and C). By contrast, leptin treatment induced only modest signaling in cells expressing the LEPR-P316T mutant, and no signaling in cells expressing the LEPR-A409E mutant. (FIG. 2, panel A). Moreover, no LEPR signaling was detected in any of the cell lines treated with vehicle or IgG control antibody (data not shown). Other signaling-defective or signaling-impaired LEPR mutants were tested in this assay but were not activated by anti-LEPR mutants (data not shown), suggesting that this rescue effect may be mutant-dependent.

The results of this Example indicate that the agonist anti-LEPR antibodies of the present invention may be useful in the treatment of diseases and disorders (e.g., early-onset obesity) that are caused by or associated with certain signaling-defective or signaling-impaired LEPR mutants (e.g., LEPR-P316T or LEPR-A409E).

### Example 9: Octet Cross-Competition Between Different Anti-LEPR Monoclonal Antibodies

Binding competition between a panel of different anti-LEPR monoclonal antibodies was determined using a real time, label-free bio-layer interferometry assay on the Octet HTX biosensor platform (Pall ForteBio Corp.). The entire experiment was performed at 25° C. in buffer containing 10 mM HEPES, 150 mM NaCl, 3 mM EDTA, and 0.05% v/v Surfactant Tween-20, 1 mg/mL BSA, pH7.4 (HBS-EBT) with the plate shaking at the speed of 1000 rpm. To assess whether two antibodies were able to compete with one another for binding to their respective epitopes on recombinant human LEPR expressed with a C-terminal myc-myc-hexahistidine tag (hLEPR.mmh; SEQ ID NO: 114), around 0.25 nm or 0.34 nm of hLEPR-MMH was first captured onto anti-penta-His antibody coated Octet biosensor tips (Fortebio Inc, #18-5122) by submerging the biosensor tips for 5 minutes in wells containing 20 μg/mL of hLEPR-MMH. The antigen captured biosensor tips were then saturated with a first anti-LEPR monoclonal antibody (subsequently referred to as mAb-1) by dipping into wells containing 50 μg/mL solution of mAb-1 for 210 seconds. The biosensor tips were then subsequently dipped into wells containing a 50 μg/mL solution of a second anti-LEPR monoclonal antibody (subsequently referred to as mAb-2) for 150 seconds. The biosensor tips were washed in HBS-EBT buffer in between every step of the experiment. The real-time binding response was monitored during the entire course of the experiment and the binding response at the end of every step was recorded. The response of mAb-2 binding to hLEPR-MMH pre-complexed with mAb-1 was compared and competitive/non-competitive behavior of different anti-LEPR monoclonal antibodies was determined as shown in Table 14 and Table 15.

TABLE 14
Cross-competition between anti-LEPR monoclonal antibodies

| First antibody | Second antibody |
|---|---|
| (mAb-1) binding to | (mAb-2) shown to |
| captured hLEPR-MMH | compete with mAb-1 |
| H4H18492P2 | H4H18417P2 |
| | H4H18438P2 |
| H4H18417P2 | H4H18492P2 |
| | H4H18438P2 |
| H4H18438P2 | H4H18492P2 |
| | H4H18417P2 |
| H4H16650P2 | H4H16679P2 |
| H4H16679P2 | H4H16650P2 |
| H4H18445P2 | H4H18482P2 |
| | H4H18487P2 |
| | H4H18446P2 |
| H4H18446P2 | H4H18482P2 |
| | H4H18487P2 |
| | H4H18445P2 |
| H4H18482P2 | H4H18445P2 |
| | H4H18487P2 |
| H4H18487P2 | H4H18445P2 |
| | H4H18482P2 |
| H4H18449P2 | None |
| Comparator Antibody | None |

TABLE 15
Cross-competition between anti-LEPR monoclonal antibodies

| | mAb-2 that competes |
|---|---|
| mAb-1 | with mAb-1 |
| H4H17319P2 | H4H17321P2 |
| | H4H16650P2 |
| | H4H16679P2 |
| H4H17321P2 | H4H17319P2 |
| | H4H16650P2 |
| | H4H16679P2 |
| H4H16650P2 | H4H17319P2 |
| | H4H17321P2 |
| | H4H16679P2 |
| H4H16679P2 | H4H17319P2 |
| | H4H17321P2 |
| | H4H16650P2 |

### Example 10: In vivo efficacy of LEPR agonist antibodies H4H16650P2, H4H16679P2, H4H17319P2 and H4H17321P2 in an Inducible Mouse Model of Leptin Deficiency

The effects of four specific agonist anti-LEPR antibodies of the invention, H4H16650P2, H4H16679P2, H4H17319P2, and H4H17321P2 on food intake, body weight and adiposity were determined in an inducible model of leptin deficiency in genetically engineered LEPR^{Hu/Hu} mice, that express a leptin receptor which is composed of the human LEPR ectodomain sequence in place of the murine LEPR ectodomain sequence. The model of leptin deficiency was induced by hydrodynamic DNA delivery (HDD) of a plasmid encoding an hFc-tagged mouse LEPR ectodomain (referred to herein as mLEPR.hFc or “Leptin trap”; SEQ ID NO: 120). The Leptin trap when expressed is secreted and binds circulating Leptin. After HDD of 50 μg of the DNA construct encoding the Leptin trap, mice exhibited increased food consumption and increased adiposity and body weight.

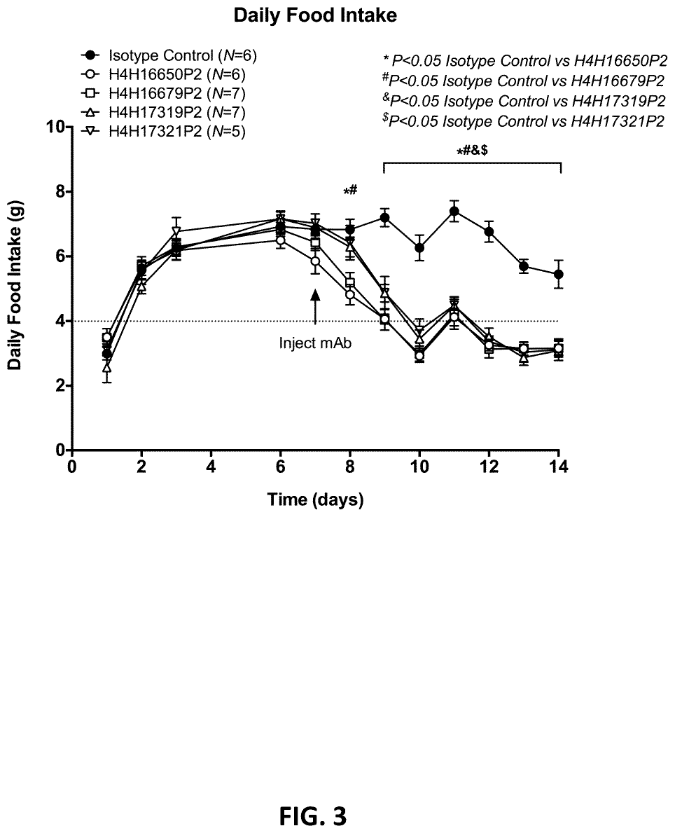

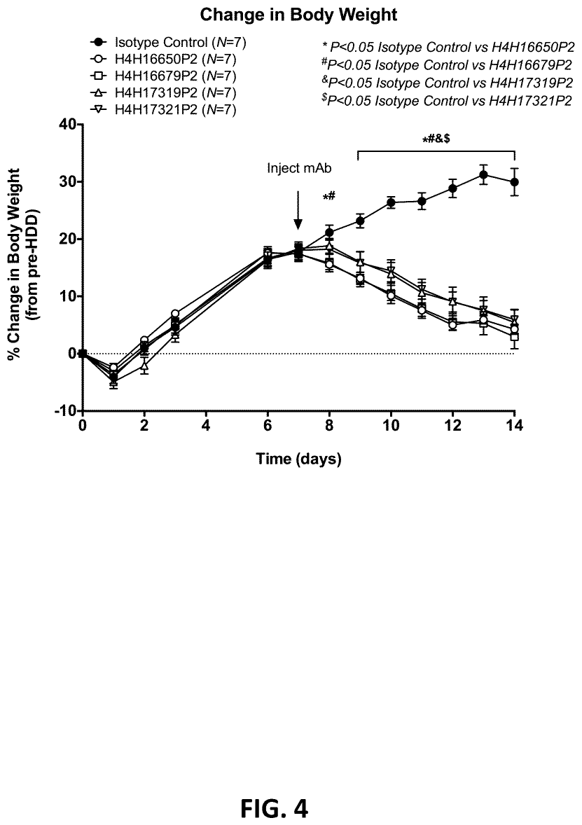

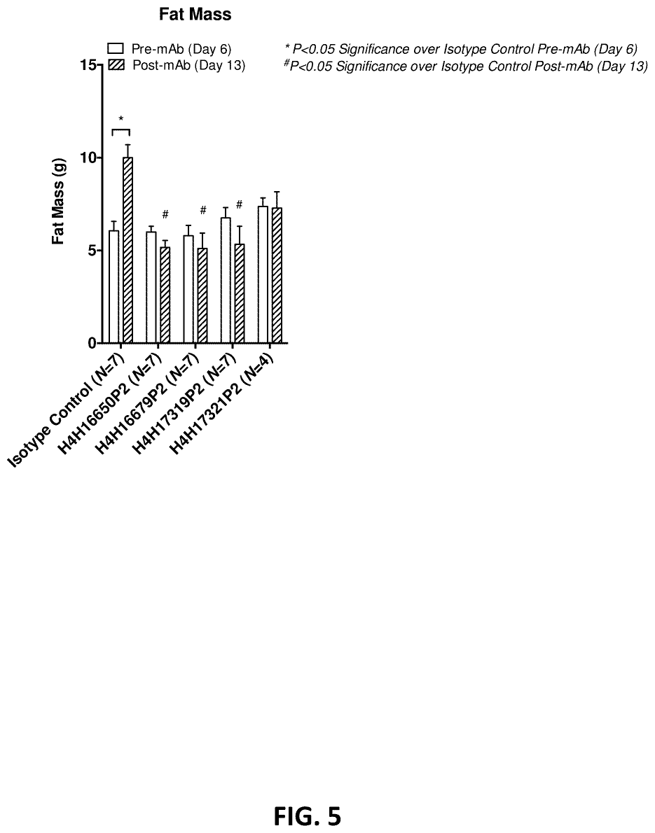

Baseline daily food intake was measured between 7 and 4 days prior to administration of the Leptin trap (days −7 and −4). On day 0, thirty-five 13- to 17-week old male LEPR^{Hu/Hu} mice were successfully subjected to HDD with the Leptin trap. On days 6 and 13 post HDD, retro-orbital bleeds were collected and body composition including adiposity was quantified by μCT. On day 7 post HDD, mice were randomized into five groups of 7 mice based on percent body weight change from day 0. Each group received via subcutaneous injection either a single dose of isotype control antibody at 3 mg/kg, H4H16650P2 at 3 mg/kg, H4H16679P2 at 3 mg/kg, H4H17319P2 at 3 mg/kg, or H4H17321 at 3 mg/kg. The isotype control antibody did not bind any known mouse protein. Food intake and body weight were measured for each animal for the duration of the study. FIG. 3 summarizes the average daily food intake for each treatment group. In FIG. 3, the dotted line represents the average baseline food intake prior to HDD injection. The percent change in body weight from day 0 was calculated for each animal at each time point. FIG. 4 summarizes the average percent change in body weight for animals in each antibody treatment group. FIG. 5 summarizes the average fat mass for animals in each antibody treatment group quantified by μCT 1 day prior to and 6 days following antibody treatment. All results are expressed as mean±SEM.

As shown in FIGS. 3 and 4, following HDD with the Leptin trap, similar increases in food intake and percent change in body weight were observed among the groups of mice before antibody treatment. As shown in FIG. 3, mice treated with antibodies H4H16650P2 or H4H16679P2 at 3 mg/kg exhibited significant reductions in food intake starting at one day after antibody treatment (day 8 post HDD) and at subsequent time points measured as compared to mice injected with the isotype control antibody. Mice treated with antibodies H4H17319P2 or H4H17321P2 at 3 mg/kg exhibited a significant reduction in food intake at two days post antibody treatment (day 9 post HDD) and at the other subsequent time points measured as compared to mice injected with isotype control antibody. As shown in FIG. 4, mice treated with antibody H4H16650P2 at 3 mg/kg exhibited a significant reduction in percent body weight change one day after antibody treatment (day 8 post HDD) and at other subsequent time points measured as compared to mice injected with isotype control antibody. One day after antibody treatment, on day 8, mice treated with the isotype control showed a 21.16±1.27% increase in body weight from day 0, whereas mice treated with H4H16650P2 had a 15.57±0.9% increase in body weight from day 0. Mice treated with antibodies H4H16679P2, H4H17319P2 or H4H17321P2 at 3 mg/kg exhibited a significant reduction in percent body weight change two days after antibody treatment (day 9 post HDD) and at other subsequent time points measured as compared to mice injected with isotype control antibody. On day 9, the % body weight changes from day 0 were 23.18±1.22, 13.17±1.05, 12.95±1.26, 15.98±1.78 and 15.83±2.01 for mice treated with isotype control, H4H16650P2, H4H16679P2, H4H17319P2, or H4H17321P2, respectively. As shown in FIG. 5, mice treated with isotype control antibody at 3 mg/kg demonstrated a significant increase in fat mass 6 days after antibody treatment (day 13 post HDD) as compared to 1 day prior to antibody treatment (day 6 post HDD). Mice treated with antibodies H4H16650P2, H4H16679P2, H4H17319P2, or H4H17321P2 at 3 mg/kg did not gain adipose mass after antibody treatment as compared to pre-antibody treatment. After 6 days of treatment (day 13 post HDD), mice treated with antibodies H4H16650P2, H4H16679P2 or H4H17319P2 at 3 mg/kg demonstrated significant decreases in fat mass as compared to mice treated with isotype control antibody at 3 mg/kg.

### Example 11: Epitope Mapping of H4H16650P2 Binding to Human Leptin Receptor (hLEPR.mmh) by Hydrogen Deuterium Exchange

Experiments were conducted to determine the amino acid residues of hLEPR.mmh (amino acids M1-D839 of SEQ ID NO: 114) with which H4H16650P2 interacts. For this purpose H/D exchange epitope mapping with mass spectrometry was carried out. A general description of the H/D exchange method is set forth in, e.g., Ehring (1999) Analytical Biochemistry 267 (2): 252-259; and Engen and Smith (2001) Anal. Chem. 73: 256A-265A.

Experimental procedure. HDX-MS experiments were performed on an integrated Waters HDX/MS platform, consisting of a Leaptec HDX PAL system for the deuterium labeling, a Waters Acquity M-Class (Auxiliary solvent manager) for the sample digestion and loading, a Waters Acquity M-Class (μBinary solvent manager) for the analytical column gradient, and Synapt G2-Si mass spectrometer for peptic peptide mass measurement.

The labeling solution was prepared in 10 mM PBS buffer in D20 at pD 7.0 (equivalent to pH 6.6). For deterium labeling, 3.8 μL of hLEPR.mmh (8 pmol/μL) or hLEPR.mmh premixed with the antibody in 2:1 molar ratio was incubated with 56.2 μL D20 labeling solution for various time-points (e.g., undeuterated control=0 sec, labeled for 1 min and 20 min). The deuteration was quenched by transferring 50 μL sample to 50 μL pre-chilled quench buffer (0.2 M TCEP, 6 M guanidine chloride in 100 mM phosphate buffer, pH 2.5) and the mixed sample was incubated at 1.0° C. for two minutes. The quenched sample was then injected into a Waters HDX Manager for online pepsin/protease XIII digestion. The digested peptides were trapped onto an ACQUITY UPLC BEH C18 1.7-μm, 2.1×5 mm VanGuard pre-column at 0° C. and eluted to an analytical column ACQUITY UPLC BEH C18 1.7-μm, 1.0×50 mm for a 9-minute gradient separation of 5%-40% B (mobile phase A: 0.1% formic acid in water, mobile phase B: 0.1% formic acid in acetonitrile). The mass spectrometer was set at cone voltage of 37 V, scan time of 0.5 s, and mass/charge range of 50-1700 Th.

For the identification of the peptides from human LEPR, LC-MSE data from undeuterated sample were processed and searched against the database including human LEPR, pepsin, and their randomized sequences via Waters ProteinLynx Global Server (PLGS) software. The identified peptides were imported to DynamX software and filtered by two criteria: 1) minimum products per amino acid: 0.2, and 2) replication file threshold: 3. DynamX software then automatically determined deuterium uptake of each peptide based on retention time and high mass accuracy (<10 ppm) across multiple time points with 3 replicates at each time.

Results. Using the online pepsin/protease XIII column coupled with MSE data acquisition, total 201 peptides from human LEPR were reproducibly identified in the absence or presence of the antibody, representing 70% sequence coverage. Five peptides had significantly reduced deuteration uptake (centroid delta values >0.4 daltons with p-values <0.05) when bound to H4H16650P2 as shown in the Table 16. The recorded peptide mass corresponds to the average value of the centroid MH+mass from three replicates. These peptides, corresponding to amino acids 162-169 (amino acids LYVLPEVL of human LEPR; SEQ ID NO: 113), and to amino acids 170-181 (amino acids EDSPLVPQKGSF of human LEPR; SEQ ID NO: 113), had a slower deuteration rate when bound to H4H16650P2. These identified residues also correspond to residues acids 162-169 and 170-181 of human LEPR as defined by Uniprot entry P48357 (SEQ ID NO. 113; Human leptin receptor)

TABLE 16
Human Leptin receptor peptides with significant
protection upon binding to antibody H4H16650P2

| | 1 min Deuteration | | | 20 min Deuteration | | |
|---|---|---|---|---|---|---|
| | | hLEPR.mmh + | | | hLEPR.mmh + | |
| Residues | hLEPR.mmh | H4H16650P2 | Δ | hLEPR.mmh | H4H16650P2 | Δ |
| 162-169 | 949.03 ± 0.03 | 947.99 ± 0.02 | −1.04 | 949.23 ± 0.02 | 948.16 ± 0.02 | −1.03 |
| 163-169 | 835.82 ± 0.03 | 834.79 ± 0.02 | −1.03 | 836.03 ± 0.02 | 834.94 ± 0.02 | −1.08 |
| 170-181 | 1310.02 ± 0.05 | 1309.12 ± 0.03 | −0.89 | 1309.77 ± 0.02 | 1309.38 ± 0.02 | −0.39 |

### Example 12: In Vivo Efficacy Testing of LEPR Potentiator Antibodies in Humanized LEPR Mice

The effects of three specific potentiator anti-LEPR antibodies of the invention, H4H18482P2, H4H18487P2 and H4H18492P2, on body weight and adiposity were determined in singly-housed genetically engineered LEPR^{Hu/Hu} mice, that express a leptin receptor which is composed of the human LEPR ectodomain sequence in place of the murine LEPR ectodomain sequence (mLEPR.hFc, SEQ ID NO: 120).

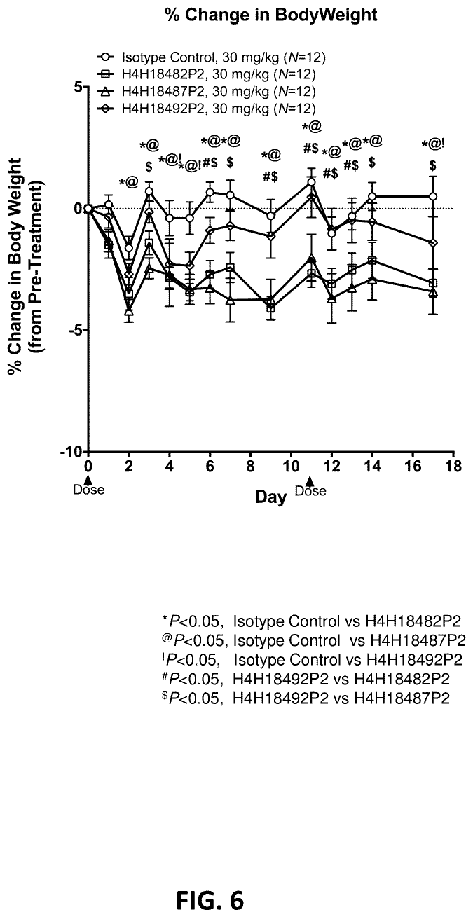

On days −19 body composition including adiposity was quantified by μCT. On days 0, forty-eight 14 to 16-week old female LEPR^{Hu/Hu} mice were randomized to four groups of 12 mice based on body weight. On days 0 and 11, mice from each group received via subcutaneous injection a single dose of isotype control antibody at 30 mg/kg, H4H18482P2 at 30 mg/kg, H4H18487P2 at 30 mg/kg or H4H18492P2 at 30 mg/kg. The isotype control antibody does not bind any known mouse protein. Body weight was measured for the duration of the study for each animal. The percent change in body weight from day 0 was calculated for each animal at each time point. FIG. 6 summarizes the average percent change in body weight for animals in each treatment group. FIG. 6 summarizes the average fat mass for animals in each antibody treatment group quantified by μCT 19 days prior to and 11 days following antibody treatment. All results are expressed as mean±SEM.

As shown in FIG. 6, decreases in percent change in body weight were observed following dosing with the LEPR potentiator antibodies, but not the isotype control antibody. As shown in FIG. 6, mice treated with H4H18482P2 at 30 mg/kg exhibited significant decreases in percent body weight change starting two days after treatment (day 2), and at the other time points compared to mice injected with an isotype control antibody. Mice treated with H4H18487P2 at 30 mg/kg exhibited significant decreases in percent body weight change starting at day 2 and at the other time points compared to mice injected with isotype control antibody. Mice treated with H4H18492P2 at 30 mg/kg exhibited a significant reduction in percent body weight change on days 4, 5 and 17 but not at other time points compared to mice injected with isotype control antibody. Mice treated with H4H18482P2 at 30 mg/kg exhibited a significant decrease in percent body weight change starting at day 6 and on subsequent days but not days 7, 14 and 17, compared to mice injected with H4H18492P2. Mice treated with H4H18487P2 at 30 mg/kg exhibited a significant decrease in percent body weight change starting at day 3 and at the other time points, but not days 4 and 5, compared to mice injected with H4H18492P2.

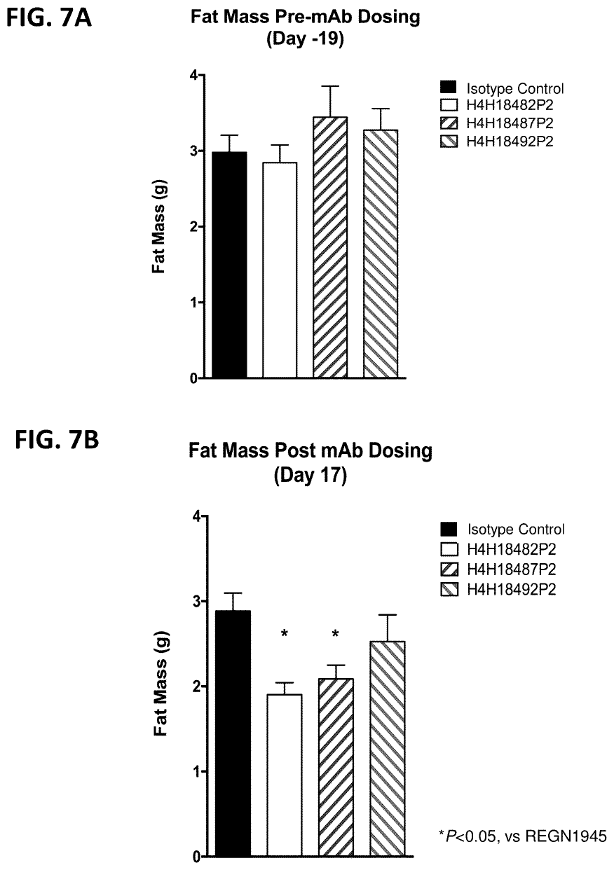

As shown in FIG. 7A, there were no differences in fat mass between the groups prior to treatment (day-19). As shown in FIG. 7B, mice treated with antibodies H4H18482 and H4H18487, but not H4H18492, at 30 mg/kg showed a statistically significant decrease in fat mass 17 days after treatment (day 12) as compared to the isotype control antibody.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### Example 13: Effect of Anti-LEPR Antibodies of the Invention on Monkey LEPR Signaling

In order to assess transcriptional activation of monkey Leptin receptor, a stable cell line was developed. IMR-32 cells (human Neuroblastoma ATCC) were generated to stably express the extracellular domain of Macaca fascicularis LEPR (MfLEPR; amino acids 22 through 837 of accession number XP_005543194.1 with threonine at 827 changed to alanine) fused with the transmembrane and cytosolic domains of human LEPR (hLEPR; amino acids 840 through 1165 of accession number NP_002294.2) along with a luciferase reporter (STAT3-Luc; SABiosciences, #CLS-6028L). The resulting cell line, referred to hereafter as IMR-32/STAT3-Luc/MfLEPR was isolated and maintained in MEM-Earl medium supplemented with 10% FBS, NEAA, 1 μg/mL Puromycin, 100 μg/mL of Hygromycin B and Penicillin/Streptomycin/L-Glutamine.

The bioassay was performed to measure the effect of anti-LEPR antibodies of the invention on monkey LEPR signaling in the absence of Leptin. For the bioassay, IMR-32/STAT3-Luc/MfLEPR cells were plated at 10,000 cells/well in a 96-well plate in 0.1% FBS in Optimem with penicillin/streptomycin (assay buffer) and incubated overnight at 37° C. in 5% CO2. The following day human leptin (hLeptin), anti-LEPR antibodies or an isotype control antibody were serially diluted from 50 nM to 0.8 pM in the assay buffer (plus a sample containing buffer alone without test molecule) and added to the cells. After 5.5 hours at 37° C. in 5% CO2, luciferase activity was measured with OneGlo™ reagent (Promega, #E6031) and Victor™ X multilabel plate reader (Perkin Elmer). The results were analyzed using nonlinear regression (4-parameter logistics) with Prism™M6 software (GraphPad) to obtain EC₅₀ values. Percentage of activation of antibodies was calculated as the maximum range of RLU achieved by the antibody relative to that of maximum range of RLU achieved by hLeptin.

As shown in Table 17, in the absence of hLeptin, all of the anti-LEPR antibodies tested showed activation of monkey LEPR signaling in IMR-32/STAT3-Luc/mfLEPR cells with EC₅₀ values ranging from 266 pM to 368 pM and maximal activation ranging from 76% to 82% where 100% activation was obtained with hLeptin. hLeptin activated with an EC₅₀ value of 333 pM. The isotype control antibody did not demonstrate any measurable stimulation of the IMR-32/STAT3-Luc/mfLEPR cells.

TABLE 17
Activation of Macaca fascicularis LEPR by anti-LEPR antibodies

| | | % |
|---|---|---|
| Leptin or Antibody | EC₅₀ (M) | Activation |
| Human Leptin | 3.33E−11 | 100 |
| H4H16650P2 | 2.66E−10 | 82 |
| H4H16679P2 | 2.49E−10 | 80 |
| H4H17319P2 | 3.65E−10 | 76 |
| H4H17321P2 | 3.68E−10 | 78 |
| Isotype control antibody | No Activation | No Activation |

### Example 14: Epitope Binding to the Full-Length Extracellular Domain of Human LEPR Using Luminex MFI Signal

To determine the epitope of human LEPR on which anti-LEPR antibodies of the invention bind, a Luminex FLEXMAP (FM3DD, LuminexCorp) flow cytometry based analysis was utilized to characterize the interaction of anti-LEPR antibodies with recombinant human LEPR protein domains. For the assay, approximately 3 million carboxylated Microplex® microspheres (Luminex, Cat #LC1000A), were washed, vortexed and sonicated in 0.1 M NaPO₄, pH 6.2 (activation buffer) and then centrifuged to remove the supernatant. The microspheres were resuspended in 120 μL of activation buffer and the carboxylate groups (—COOH) were activated by addition of 15 μL of 50 mg/ml of N-hydroxysuccinimide (NHS, Thermo Scientific, Cat #24500) followed by addition of 15 μL of 50 mg/ml of 1-ethyl-3-[3-dimethylaminopropyl] carbodiimide (EDC, ThermoScientific, Cat #22980) at 25° C. After 10 minutes, the pH of the reaction was reduced to 5.0 with the addition of 600 μL of 50 mM MES, pH 5 (coupling buffer), and the microspheres were vortexed, and centrifuged to remove supernatant. The activated beads were immediately mixed with 500 μL of 20 μg/mL monoclonal anti-myc antibodies with either a mouse IgG or a human IgG, in coupling buffer and incubated for two hours at 25° C. The coupling reaction was quenched by addition of 50 μL of 1M Tris-HCl, pH 8.0 and the microspheres were rapidly vortexed, centrifuged, and washed four times with 1 mL of DPBS, to remove uncoupled proteins and other reaction components.

The transiently expressed LEPR proteins, including human LEPR extracellular domain expressed with a C-terminal myc-myc hexahistidine tag (human LEPR-MMH, SEQ ID NO: 113), human LEPR CRH1 (D1) expressed with a C-terminal myc-myc hexahistidine tag (human LEPR CRH1 (D1)-MMH, amino acids 1-208 of SEQ ID NO: 113 with a myc-myc hexahistidine tag, amino acids 209-236), human LEPR CRH1 (D1,D2) domain expressed with a C-terminal myc-myc hexahistidine tag (human LEPR CRH1 (D1,D2)-MMH, amino acids 1-318 of SEQ ID NO: 113 with a myc-myc hexahistidine tag, amino acids 319-346), human LEPR CRH1-Ig (D1,D2,D3) domain expressed with a C-terminal myc-myc hexahistidine tag (human LEPR CRH1 (D1,D2,D3)-MMH, amino acids 1-278 of SEQ ID NO: 113 with a myc-myc hexahistidine tag, amino acids 279-306), human LEPR CRH1-Ig (D2,D3) domain expressed with a C-terminal myc-myc hexahistidine tag (human LEPR CRH1-Ig (D2,D3)-MMH, amino acids 1-198 of SEQ ID NO: 113 with a myc-myc hexahistidine tag, amino acids 199-226), human LEPR Ig (D3) domain expressed with a C-terminal myc-myc hexahistidine tag (human LEPR Ig (D3)-MMH, amino acids 1-88 of SEQ ID NO: 113 with a myc-myc hexahistidine tag, amino acids 89-116), human LEPR CRH2 domain expressed with a C-terminal myc-myc hexahistidine tag (human LEPR CRH2-MMH, amino acids 1-207 of SEQ ID NO: 113 with a myc-myc-hexahistidine tag, amino acids 208-235), human LEPR FNIII domain expressed with a C-terminal myc-myc hexahistidine tag (human LEPR FNIII-MMH, amino acids 1-204 of SEQ ID NO: 113 with a myc-myc hexahistidine tage, amino acids 205-232), and human LEPR Ig-CRH2-FNIII domain expressed with a C-terminal myc-myc hexahistidine tag (human LEPR Ig-CRH2-FNIII-MMH, amino acids 1-510 of SEQ ID NO: 113 with a myc-myc-hexahistidine tag, amino acids 511-538), were suspended in serum free CHO-S-SFM II Medium (Thermo Fisher, Cat #31033020) and were then clarified by centrifugation. Aliquots of microspheres with immobilized anti-myc monoclonal antibodies, prepared as described above, were added individually to 1 mL of the each of these protein supernatants. The microspheres were gently mixed, incubated for two hours at 25° C., washed twice with 1 mL of DBPS, centrifuged to remove the supernatant and finally resuspended in 1 mL of DPBS buffer. Forty-eight μL of anti-myc IgG coupled microspheres from individual reactions with full length human LEPR and with each of the human LEPR domain proteins were withdrawn and mixed together in 3.6 mL of PBS+20 mg/mL BSA+0.05% sodium azide (blocking buffer).

From this mixed pool, 75 μL of microspheres were plated per well on a 96 well filter plate (Millipore, Cat. No: MSBVN1250) and mixed with 25 μL of individual anti-human LEPR monoclonal antibodies (0.5 or 5 g/mL), incubated for two hours at 25° C. and then washed twice with 200 μL of DPBS with 0.05% Tween 20 (washing buffer). To detect and quantify the amounts of bound anti-LEPR antibody levels to individual microspheres, either 100 μL of 2.5 μg/mL R-Phycoerythrin conjugated goat F(ab′)₂ anti-human kappa (Southern Biotech, Cat #2063-09) in blocking buffer or 100 μL of 1.25 μg/mL R-Phycoerythrin AffiniPure F(ab′)2 Fragment Goat Anti-Mouse IgG, F(ab′)2 Fragment Specific (Jackson Immunoresearch, Cat. No: 115-116-072) in blocking buffer, was added and incubated for 30 minutes at 25° C. After 30 minutes, the samples were washed twice with 200 μL of washing buffer and resuspended in 150 μL of wash buffer. The Median Fluorescence intensity (MFI) of the microspheres was measured in a Luminex Analyzer.

TABLE 18
Luminex MFI signal of anti-LEPR antibodies binding to myc tag captured full-
length extracellular domain of human LEPR and isonalted human LEPR domains

| | | | | | | | | | Full Length | |
|---|---|---|---|---|---|---|---|---|---|---|
| | CRH1 | CRH1 | CRH1-Ig | CRH1-Ig | Ig | | | Ig-CRH2- | extracellular | Probable |
| Antibody | (D1) | (D1, D2) | (D1, D2, D3) | (D2, D3) | (D3) | CRH2 | FNIII | FNIII | domain | Binding site |
| H4H18445P2 | 12 | 30 | 22 | 40 | 19 | 17 | 230 | 14544 | 6573 | FNIII |
| H4H18446P2 | 17 | 682 | 205 | 645 | 25 | 65 | 32 | 16852 | 10536 | Ig-CRH2- |
| | | | | | | | | | | FNIII |
| H4H18482P2 | 13 | 40 | 21 | 52 | 27 | 23 | 167 | 15316 | 7311 | Ig-CRH2- |
| | | | | | | | | | | FNIII |
| H4H18487P2 | 12 | 51 | 29 | 62 | 22 | 27 | 174 | 16320 | 7329 | Ig-CRH2- |
| | | | | | | | | | | FNIII |
| H4H18417P2 | 10 | 16048 | 3334 | 5502 | 17 | 39 | 14 | 37 | 4887 | CRH1 D2 |
| H4H18438P2 | 13 | 18931 | 6572 | 8884 | 30 | 165 | 25 | 468 | 6251 | CRH1 D2 |
| H4H18492P2 | 11 | 19371 | 6354 | 8685 | 19 | 18 | 16 | 186 | 6382 | CRH1 D2 |
| H4H18449P2 | 20 | 2934 | 2056 | 42 | 24 | 15 | 13 | 43 | 7976 | CRH1(D1-2) |
| H4H16650P2 | 8 | 4722 | 2562 | 74 | 10 | 16 | 6 | 110 | 7603 | CRH1(D1-2) |
| H4H16679P2 | 12 | 4388 | 2797 | 34 | 14 | 33 | 10 | 42 | 7507 | CRH1(D1-2) |
| H4H17319P2 | 8 | 1246 | 938 | 14 | 8 | 91 | 20 | 8 | 3305 | CRH1(D1-2) |
| H4H17321P2 | 9 | 2649 | 1752 | 15 | 7 | 116 | 40 | 14 | 4696 | CRH1(D1-2) |
| Comparator | −14 | 19 | −57 | 27 | 10 | 9404 | 73 | 7112 | 3908 | CRH2 |
| mAb | | | | | | | | | | |

The results of the Luminex based analysis are tabulated in Table 18. Luminex MFI signal intensities indicate that the twelve anti-LEPR antibodies of the invention bound to the complete human LEPR extracellular domain. Anti-LEPR antibodies H4H18417P2, H4H18438P2, and H4H18492P2, bound to epitopes within the CRH1 D2 domain of human LEPR. Anti-LEPR antibodies H4H18449P2, H4H16650P and H4H16679P, bound to epitopes within the CRH1 (D1-2) domain of human LEPR. Anti-LEPR antibody Comparator mAB, bound to epitopes within the CRH2 domain of human LEPR. Anti-LEPR antibody H4H18445P2 bound to epitopes within the FNIII domain of human LEPR. Anti-LEPR antibodies H4H18446P2, H4H18482P2 and H4H18487P2, bound to epitopes within the Ig-CRH2-FNIII domain of human LEPR.

TABLE 19
Informal Sequence Listing

| SEQ. ID. | | |
|---|---|---|
| NO. | Sequence | Description |
| 1 | caggtgcagc tggtggagtc tgggggaggc gtggtccagc ctgggaagtc cctgagactc | DNA |
| | tcctgtgtag cgtctggatt caccttcagt tccgatgcca tgtactgggt ccgccaggct | nucleotide |
| | ccaggcaagg ggctggaatg ggtggcagtt atttattatg atggaaatta tcaatactat | sequence |
| | gaagactccg ttaagggtcg attcaccatc tccagagaca attcccagaa cacgctggat | |
| | ctgcaaatga acagcctgag agtcgacgac acggctgtat atttctgtgc gcgtctcaac | |
| | tgggattact ggtatctcga tctctggggc cgtggcaccc tggtcactgt ctcctca | |
| 2 | QVQLVESGGG VVQPGKSLRL SCVASGFTFS SDAMYWVRQA | Amino Acid |
| | PGKGLEWVAV IYYDGNYQYY EDSVKGRFTI SRDNSQNTLD | sequence |
| | LQMNSLRVDD TAVYFCARLN WDYWYLDLWG RGTLVTVSS | |
| 3 | ggattcacct tcagttccga tgcc | DNA |
| | | nucleotide |
| | | sequence |
| 4 | GFTFSSDA | Amino Acid |
| | | sequence |
| 5 | atttattatg atggaaatta tcaa | DNA |
| | | nucleotide |
| | | sequence |
| 6 | IYYDGNYQ | Amino Acid |
| | | sequence |
| 7 | gcgcgtctca actgggatta ctggtatctc gatctc | DNA |
| | | nucleotide |
| | | sequence |
| 8 | ARLNWDYWYL DL | Amino Acid |
| | | sequence |
| 9 | gacatccaga tgacccagtc tccatcctcc ctgtctgcat ctgtaggaga cagagtcacc | DNA |
| | atcacttgcc gggcaagtca gagcattagc agctatttaa attggtatca gcagaaacca | nucleotide |
| | gggaaagccc ctaagctcct gatctatgct gcatccagtt tgcaaagtgg ggtcccgtca | sequence |
| | aggttcagtg gcagtggatc tgggacagat ttcactctca ccatcagcag tctgcaacct | |
| | gaagattttg caacttacta ctgtcaacag agttacagta cccctccgat caccttcggc | |
| | caagggacac gactggagat taaa | |
| 10 | DIQMTQSPSS LSASVGDRVT ITCRASQSIS SYLNWYQQKP | Amino Acid |
| | GKAPKLLIYA ASSLQSGVPS RFSGSGSGTD FTLTISSLQP | sequence |
| | EDFATYYCQQ SYSTPPITFG QGTRLEIK | |
| 11 | cagagcatta gcagctat | DNA |
| | | nucleotide |
| | | sequence |
| 12 | QSISSY | Amino Acid |
| | | sequence |
| 13 | gctgcatcc | DNA |
| | | nucleotide |
| | | sequence |
| 14 | AAS | Amino Acid |
| | | sequence |
| 15 | caacagagtt acagtacccc tccgatcacc | DNA |
| | | nucleotide |
| | | sequence |
| 16 | QQSYSTPPIT | Amino Acid |
| | | sequence |
| 17 | caggtgcagc tggtggagtc cgggggaggc gtggtccagc ctgggaggtc | DNA |
| | cctgagactc tcctgtacag cgtctggatt caccttcagt agttatgcca tgtactgggt | nucleotide |
| | ccgccaggct ccaggcaagg ggctggagtg ggtgtcagtt atatactatg atggaagtta | sequence |
| | taaatactat gcagactccg tgaagggccg attcaccatc tccagagaca attccaagaa | |
| | cacgctgtat ctgcaaatgg acagcctgag agccgaggac acggctgtct attactgtgc | |
| | gagttataac tggaactact ggtacttcga tttctggggc cgtggcaccc tggtcactgt | |
| | ctcctca | |
| 18 | QVQLVESGGG VVQPGRSLRL SCTASGFTFS SYAMYWVRQA | Amino Acid |
| | PGKGLEWVSV IYYDGSYKYY ADSVKGRFTI SRDNSKNTLY | sequence |
| | LQMDSLRAED TAVYYCASYN WNYWYFDFWG RGTLVTVSS | |
| 19 | ggattcacct tcagtagtta tgcc | DNA |
| | | nucleotide |
| | | sequence |
| 20 | GFTFSSYA | Amino Acid |
| | | sequence |
| 21 | atatactatg atggaagtta taaa | DNA |
| | | nucleotide |
| | | sequence |
| 22 | IYYDGSYK | Amino Acid |
| | | sequence |
| 23 | gcgagttata actggaacta ctggtacttc gatttc | DNA |
| | | nucleotide |
| | | sequence |
| 24 | ASYNWNYWYF DF | Amino Acid |
| | | sequence |
| 25 | caggtgcagc tggtggagtc tgggggaagc gtggtccagc ctgggaggtc cctgagactc | DNA |
| | tcctgtgcag cgtctggatt caccttcagt acatatgcca tgtactgggt ccgccagact | nucleotide |
| | ccaggcaagg ggctggagtg ggtggctgtt ttatactctg atggaagtaa taaatactat | sequence |
| | atagactccg tgaagggccg attcaccatc tccagagaca cttccacgaa cactctgtat | |
| | ctgcaaatga gcagcctgcg agccgacgac tcggctctat attactgtgc gcgtctcaac | |
| | tgggattact ggtacttcga tctctggggc cgtggcaccc tggtcactgt ctcctca | |
| 26 | QVQLVESGGS VVQPGRSLRL SCAASGFTFS TYAMYWVRQT | Amino Acid |
| | PGKGLEWVAV LYSDGSNKYY IDSVKGRFTI SRDTSTNTLY | sequence |
| | LQMSSLRADD SALYYCARLN WDYWYFDLWG RGTLVTVSS | |
| 27 | ggattcacct tcagtacata tgcc | DNA |
| | | nucleotide |
| | | sequence |
| 28 | GFTFSTYA | Amino Acid |
| | | sequence |
| 29 | ttatactctg atggaagtaa taaa | DNA |
| | | nucleotide |
| | | sequence |
| 30 | LYSDGSNK | Amino Acid |
| | | sequence |
| 31 | gcgcgtctca actgggatta ctggtacttc gatctc | DNA |
| | | nucleotide |
| | | sequence |
| 32 | ARLNWDYWYF DL | Amino Acid |
| | | sequence |
| 33 | caggtgcagc tggtggagtc tgggggaggc gtggtccagc ctgggaggtc cctgagactc | DNA |
| | tcctgtgaag cgtctggatt cagcagcagt gacaatgcca tgtactgggt ccgccaggct | nucleotide |
| | ccaggcaagg ggctggagtg ggtgtcagtt atatatcatg atggaagtta taaatactat | sequence |
| | gaagactccg tgaagggccg attcaccatc gccagagaca attccaagaa cacgctttat | |
| | ttgcaaatga acagcctgag agccgaggac acggctgtat attactgtgc gaggtataac | |
| | tggaaccact ggtacttcga tgtctggggc cgtggcaccc tggtcactgt ctcctca | |
| 34 | QVQLVESGGG VVQPGRSLRL SCEASGFSSS DNAMYWVRQA | Amino Acid |
| | PGKGLEWVSV IYHDGSYKYY EDSVKGRFTI ARDNSKNTLY | sequence |
| | LQMNSLRAED TAVYYCARYN WNHWYFDVWG RGTLVTVSS | |
| 35 | ggattcagca gcagtgacaa tgcc | DNA |
| | | nucleotide |
| | | sequence |
| 36 | GFSSSDNA | Amino Acid |
| | | sequence |
| 37 | atatatcatg atggaagtta taaa | DNA |
| | | nucleotide |
| | | sequence |
| 38 | IYHDGSYK | Amino Acid |
| | | sequence |
| 39 | gcgaggtata actggaacca ctggtacttc gatgtc | DNA |
| | | nucleotide |
| | | sequence |
| 40 | ARYNWNHWYF DV | Amino Acid |
| | | sequence |
| 41 | caggtgcagc tggtggagtc tgggggaggc gtggtccagc ctgggaggtc cctgagactc | DNA |
| | tcctgtgcag cctctggatt caccttcagt acctatggca tgcactgggt ccgccaggct | nucleotide |
| | ccaggcaagg ggctggagtg ggtgtcagtt atatcatatg acgaaagtaa taagtactat | sequence |
| | gcagactccg tgaagggccg attcaccatt tctagagaca attccaagaa cgcgctgtat | |
| | ttacaaatga acagcctgag aaatgaggac acggctgtgt attactgtgc gagagatcgg | |
| | ccttttggat tggttaccgg atggttcgac ccctggggcc agggaaccct ggtcaccgtc | |
| | tcctca | |
| 42 | QVQLVESGGG VVQPGRSLRL SCAASGFTFS TYGMHWVRQA | Amino Acid |
| | PGKGLEWVSV ISYDESNKYY ADSVKGRFTI SRDNSKNALY | sequence |
| | LQMNSLRNED TAVYYCARDR PFGLVTGWED PWGQGTLVTV | |
| | SS | |
| 43 | ggattcacct tcagtaccta tggc | DNA |
| | | nucleotide |
| | | sequence |
| 44 | GFTFSTYG | Amino Acid |
| | | sequence |
| 45 | atatcatatg acgaaagtaa taag | DNA |
| | | nucleotide |
| | | sequence |
| 46 | ISYDESNK | Amino Acid |
| | | sequence |
| 47 | gcgagagatc ggccttttgg attggttacc ggatggttcg acccc | DNA |
| | | nucleotide |
| | | sequence |
| 48 | ARDRPFGLVT GWFDP | Amino Acid |
| | | sequence |
| 49 | caggtgcagc tggtggagtc tgggggaggc gtggtccagc ctgggaggtc cctgagactc | DNA |
| | tcctgtgcag cgtctggatt cagtttcaat acctatggca tgcactgggt ccgccaggct | nucleotide |
| | ccaggcaagg ggctggagtg ggtgacaatt atatggtatg atggaagtat taaatactat | sequence |
| | gcagactccg tgaagggccg attcaccatc tccagagaca attccaagaa cacgctgtat | |
| | ctgcaaatga acagcctgag agccgaggac acggctgtgt attattgtgc gagaggtgga | |
| | tatagtggct acctctactt tgactactgg ggccagggaa ccctggtcac cgtctcctca | |
| 50 | QVQLVESGGG VVQPGRSLRL SCAASGFSFN TYGMHWVRQA | Amino Acid |
| | PGKGLEWVTI IWYDGSIKYY ADSVKGRFTI SRDNSKNTLY | sequence |
| | LQMNSLRAED TAVYYCARGG YSGYLYFDYW GQGTLVTVSS | |
| 51 | ggattcagtt tcaataccta tggc | DNA |
| | | nucleotide |
| | | sequence |
| 52 | GFSFNTYG | Amino Acid |
| | | sequence |
| 53 | atatggtatg atggaagtat taaa | DNA |
| | | nucleotide |
| | | sequence |
| 54 | IWYDGSIK | Amino Acid |
| | | sequence |
| 55 | gcgagaggtg gatatagtgg ctacctctac tttgactac | DNA |
| | | nucleotide |
| | | sequence |
| 56 | ARGGYSGYLY FDY | Amino Acid |
| | | sequence |
| 57 | caggtgcagc tgcaggagtc gggcccagga ctggtgaagc cttcacagac cctgtccctc | DNA |
| | acctgcactg tctctggtgg ctccatcagc agcggtggtg actactggag ctggatccgc | nucleotide |
| | cagctcccag ggaagggcct ggagtggatt gggtacatct attacagtgg gagcgcctac | sequence |
| | tataatccgt ccctcaagag tcgaggtacc atatcaatag acacgtctaa gaaccagttc | |
| | tccctgaagc tgacctctgt gactgccgcg gacacggccg tatatttctg tgtgaaatta | |
| | cgatttttgg agtggttctt ggggggctgg ttcggcccct ggggccaggg aaccctggtc | |
| | accgtctcct ca | |
| 58 | QVQLQESGPG LVKPSQTLSL TCTVSGGSIS SGGDYWSWIR | Amino Acid |
| | QLPGKGLEWI GYIYYSGSAY YNPSLKSRGT ISIDTSKNQF | sequence |
| | SLKLTSVTAA DTAVYFCVKL RFLEWFLGGW FGPWGQGTLV | |
| | TVSS | |
| 59 | ggtggctcca tcagcagcgg tggtgactac | DNA |
| | | nucleotide |
| | | sequence |
| 60 | GGSISSGGDY | Amino Acid |
| | | sequence |
| 61 | atctattaca gtgggagcgc c | DNA |
| | | nucleotide |
| | | sequence |
| 62 | IYYSGSA | Amino Acid |
| | | sequence |
| 63 | gtgaaattac gatttttgga gtggttcttg gggggctggt tcggcccc | DNA |
| | | nucleotide |
| | | sequence |
| 64 | VKLRFLEWFL GGWFGP | Amino Acid |
| | | sequence |
| 65 | gaggtgcagc tggtggagtc tgggggaggc ttggtacagc cgggggggtc | DNA |
| | cctgagactc tcctgtgcag cctctggatt cacctttagc aactatggca tgacctgggt | nucleotide |
| | ccgccaggct ccagggaagg gcctggaatg ggtctcagct attactggtg gtggtggtag | sequence |
| | cacatactac tcaaactccg tgaagggccg gttcaccatc tccagagaca attccaagaa | |
| | cacggtgtat ctgcgaatga acagtgtgag agccgaggac acggccgtat attactgtgc | |
| | gaaatataag tggaacttcg tggacgactg gggccaggga accacggtca ccgtctcctc | |
| | a | |
| 66 | EVQLVESGGG LVQPGGSLRL SCAASGFTFS NYGMTWVRQA | Amino Acid |
| | PGKGLEWVSA ITGGGGSTYY SNSVKGRFTI SRDNSKNTVY | sequence |
| | LRMNSVRAED TAVYYCAKYK WNFVDDWGQG TTVTVSS | |
| 67 | ggattcacct ttagcaacta tggc | DNA |
| | | nucleotide |
| | | sequence |
| 68 | GFTFSNYG | Amino Acid |
| | | sequence |
| 69 | attactggtg gtggtggtag caca | DNA |
| | | nucleotide |
| | | sequence |
| 70 | ITGGGGST | Amino Acid |
| | | sequence |
| 71 | gcgaaatata agtggaactt cgtggacgac | DNA |
| | | nucleotide |
| | | sequence |
| 72 | AKYKWNFVDD | Amino Acid |
| | | sequence |
| 73 | gaggtgcagc tggtggagtc tgggggaggc ttggtacagc ctggggggtc cctgagactc | DNA |
| | tcctgtgttg cctctggatt caccttcaat aaatacgaca tgcactgggt ccgccaaact | nucleotide |
| | actggaaaag gtctagagtg ggtctcaggt attgatactg atggtgacac atactatcca | sequence |
| | ggctccgtga agggccgatt caccatctcc agagaaaatg ccgagaactc cctgtatctt | |
| | caaatgaacg gcctgagagt cggggacacg gctgtgtatt actgtgcaag atggccttgg | |
| | agtggtttct atggtgcttt tgatatctgg ggccaaggga caatggtcac cgtctcttca | |
| 74 | EVQLVESGGG LVQPGGSLRL SCVASGFTFN KYDMHWVRQT | Amino Acid |
| | TGKGLEWVSG IDTDGDTYYP GSVKGRFTIS RENAENSLYL | sequence |
| | QMNGLRVGDT AVYYCARWPW SGFYGAFDIW GQGTMVTVSS | |
| 75 | ggattcacct tcaataaata cgac | DNA |
| | | nucleotide |
| | | sequence |
| 76 | GFTFNKYD | Amino Acid |
| | | sequence |
| 77 | attgatactg atggtgacac a | DNA |
| | | nucleotide |
| | | sequence |
| 78 | IDTDGDT | Amino Acid |
| | | sequence |
| 79 | gcaagatggc cttggagtgg tttctatggt gcttttgata tc | DNA |
| | | nucleotide |
| | | sequence |
| 80 | ARWPWSGFYG AFDI | Amino Acid |
| | | sequence |
| 81 | caggtgcagc tgcaggagtc gggcccagga ctggtgaagc cttcacagac cctgtccctc | DNA |
| | acctgcactg tctctggtgg ctccatcagc agtggtaatt actactggaa ctggatccgc | nucleotide |
| | caacagccag gagagggcct ggagtggatt gcttacatct atcacaatgg ggtcaccaac | sequence |
| | ttcaatccgt ccctcaagag tcgacttact atatcagtag acacgtctaa gactcagttc | |
| | tccctgaagt tgaggtctgt gactgccgcg gacacggccg tttattactg tgcgagatca | |
| | ggcagctggt tcgagaactg gtacttcgat ctctggggcc gtggcaccct ggtcactgtc | |
| | tcctca | |
| 82 | QVQLQESGPG LVKPSQTLSL TCTVSGGSIS SGNYYWNWIR | Amino Acid |
| | QQPGEGLEWI AYIYHNGVTN FNPSLKSRLT ISVDTSKTQF | sequence |
| | SLKLRSVTAA DTAVYYCARS GSWFENWYFD LWGRGTLVTV SS | |
| 83 | ggtggctcca tcagcagtgg taattactac | DNA |
| | | nucleotide |
| | | sequence |
| 84 | GGSISSGNYY | Amino Acid |
| | | sequence |
| 85 | atctatcaca atggggtcac c | DNA |
| | | nucleotide |
| | | sequence |
| 86 | IYHNGVT | Amino Acid |
| | | sequence |
| 87 | gcgagatcag gcagctggtt cgagaactgg tacttcgatc tc | DNA |
| | | nucleotide |
| | | sequence |
| 88 | ARSGSWFENW YFDL | Amino Acid |
| | | sequence |
| 89 | gaaattgtgt tgacgcagtc tccaggcacc ctgtctttgt ctccagggga aagagccacc | DNA |
| | ctctcctgca gggccagtca gagtgttagc agcagctact tagcctggta ccagcagaaa | nucleotide |
| | cctggccagg ctcccaggct cctcatctat ggtgcatcca gcagggccac tggcatccca | sequence |
| | gacaggttca gtggcagtgg gtctgggaca gacttcactc tcaccatcag cagactggag | |
| | cctgaagatt ttgcagtgta ttactgtcag cagtatggta gctcaccttg gacgttcggc | |
| | caagggacca aggtggaaat caaa | |
| 90 | EIVLTQSPGT LSLSPGERAT LSCRASQSVS SSYLAWYQQK | Amino Acid |
| | PGQAPRLLIY GASSRATGIP DRFSGSGSGT DFTLTISRLE | sequence |
| | PEDFAVYYCQ QYGSSPWTFG QGTKVEIK | |
| 91 | cagagtgtta gcagcagcta c | DNA |
| | | nucleotide |
| | | sequence |
| 92 | QSVSSSY | Amino Acid |
| | | sequence |
| 93 | ggtgcatcc | DNA |
| | | nucleotide |
| | | sequence |
| 94 | GAS | Amino Acid |
| | | sequence |
| 95 | cagcagtatg gtagctcacc ttggacg | DNA |
| | | nucleotide |
| | | sequence |
| 96 | QQYGSSPWT | Amino Acid |
| | | sequence |
| 97 | caggtgcagc tgcaggagtc gggcccagga ctggtgaagc cttcggagac cctgtccctc | DNA |
| | acctgcactg tctctggtgg ctccatcagt aattcctact ggagctggat ccggcagccc | nucleotide |
| | ccagggaagg gactggagtg gattggatat gtctattccc gtgggaacac caagtacaac | sequence |
| | ccctccctca cgagtcgagt caccatgtca tttgacacgt ccaagaacca gttctccctg | |
| | aaactgaggt ctgtgaccgc cgcagacacg gccgtgtatt actgtgcgag | |
| | aagcagcagc tggtacgagg actggtactt cgatctctgg ggccgtggca ccctggtcac | |
| | tgtctcctca | |
| 98 | QVQLQESGPG LVKPSETLSL TCTVSGGSIS NSYWSWIRQP | Amino Acid |
| | PGKGLEWIGY VYSRGNTKYN PSLTSRVTMS FDTSKNQFSL | sequence |
| | KLRSVTAADT AVYYCARSSS WYEDWYFDLW GRGTLVTVSS | |
| 99 | ggtggctcca tcagtaattc ctac | DNA |
| | | nucleotide |
| | | sequence |
| 100 | GGSISNSY | Amino Acid |
| | | sequence |
| 101 | gtctattccc gtgggaacac c | DNA |
| | | nucleotide |
| | | sequence |
| 102 | VYSRGNT | Amino Acid |
| | | sequence |
| 103 | gcgagaagca gcagctggta cgaggactgg tacttcgatc tc | DNA |
| | | nucleotide |
| | | sequence |
| 104 | ARSSSWYEDW YFDL | Amino Acid |
| | | sequence |
| 105 | caggtgcagc tacagcagtg gggcgcaggg ctgtttaagc cttcggagac cctgtccctc | DNA |
| | acctgcgatg tctatggtgg gtccttcaga ggttattatt ggagttggat ccgccagccc | nucleotide |
| | ccagggaagg ggctggagtg gattggggaa atcagttata gtggtttcac caattacaac | sequence |
| | ccgtccctca agagtcgagt catcatatca atagatacgt ccaagaacca gttctccctg | |
| | aagatgagct ctgtgaccgc cgcggacacg gctgtttatt actgtgcgag agttacctat | |
| | ggttatggga cctttgatta ttggggccag ggaaccctgg tcaccgtctc ctca | |
| 106 | QVQLQQWGAG LFKPSETLSL TCDVYGGSFR GYYWSWIRQP | Amino Acid |
| | PGKGLEWIGE ISYSGFTNYN PSLKSRVIIS IDTSKNQFSL | sequence |
| | KMSSVTAADT AVYYCARVTY GYGTFDYWGQ GTLVTVSS | |
| 107 | ggtgggtcct tcagaggtta ttat | DNA |
| | | nucleotide |
| | | sequence |
| 108 | GGSFRGYY | Amino Acid |
| | | sequence |
| 109 | atcagttata gtggtttcac c | DNA |
| | | nucleotide |
| | | sequence |
| 110 | ISYSGFT | Amino Acid |
| | | sequence |
| 111 | gcgagagtta cctatggtta tgggaccttt gattat | DNA |
| | | nucleotide |
| | | sequence |
| 112 | ARVTYGYGTF DY | Amino Acid |
| | | sequence |
| 113 | MICQKFCVVL LHWEFIYVIT AFNLSYPITP WRFKLSCMPP | Amino Acid |
| | NSTYDYFLLP AGLSKNTSNS NGHYETAVEP KFNSSGTHFS | sequence |
| | NLSKTTFHCC FRSEQDRNCS LCADNIEGKT FVSTVNSLVF | |
| | QQIDANWNIQ CWLKGDLKLF ICYVESLFKN LFRNYNYKVH | |
| | LLYVLPEVLE DSPLVPQKGS FQMVHCNCSV HECCECLVPV | |
| | PTAKLNDTLL MCLKITSGGV IFQSPLMSVQ PINMVKPDPP | |
| | LGLHMEITDD GNLKISWSSP PLVPFPLQYQ VKYSENSTTV | |
| | IREADKIVSA TSLLVDSILP GSSYEVQVRG KRLDGPGIWS | |
| | DWSTPRVFTT QDVIYFPPKI LTSVGSNVSF HCIYKKENKI | |
| | VPSKEIVWWM NLAEKIPQSQ YDVVSDHVSK VTFFNLNETK | |
| | PRGKFTYDAV YCCNEHECHH RYAELYVIDV NINISCETDG | |
| | YLTKMTCRWS TSTIQSLAES TLQLRYHRSS LYCSDIPSIH | |
| | PISEPKDCYL QSDGFYECIF QPIFLLSGYT MWIRINHSLG | |
| | SLDSPPTCVL PDSVVKPLPP SSVKAEITIN IGLLKISWEK | |
| | PVFPENNLQF QIRYGLSGKE VQWKMYEVYD AKSKSVSLPV | |
| | PDLCAVYAVQ VRCKRLDGLG YWSNWSNPAY TVVMDIKVPM | |
| | RGPEFWRIIN GDTMKKEKNV TLLWKPLMKN DSLCSVQRYV | |
| | INHHTSCNGT WSEDVGNHTK FTFLWTEQAH TVTVLAINSI | |
| | GASVANFNLT FSWPMSKVNI VQSLSAYPLN SSCVIVSWIL | |
| | SPSDYKLMYF IIEWKNLNED GEIKWLRISS SVKKYYIHDH | |
| | FIPIEKYQFS LYPIFMEGVG KPKIINSFTQ DDIEKHQSDA | |
| | GLYVIVPVII SSSILLLGTL LISHQRMKKL FWEDVPNPKN | |
| | CSWAQGLNFQ KPETFEHLFI KHTASVTCGP LLLEPETISE | |
| | DISVDTSWKN KDEMMPTTVV SLLSTTDLEK GSVCISDQFN | |
| | SVNFSEAEGT EVTYEDESQR QPFVKYATLI SNSKPSETGE | |
| | EQGLINSSVT KCFSSKNSPL KDSFSNSSWE IEAQAFFILS | |
| | DQHPNIISPH LTFSEGLDEL LKLEGNFPEE NNDKKSIYYL | |
| | GVTSIKKRES GVLLTDKSRV SCPFPAPCLF TDIRVLQDSC | |
| | SHFVENNINL GTSSKKTFAS YMPQFQTCST QTHKIMENKM | |
| | CDLTV | |
| 114 | FNLSYPITPW RFKLSCMPPN STYDYFLLPA GLSKNTSNSN | Amino Acid |
| | GHYETAVEPK FNSSGTHFSN LSKTTFHCCF RSEQDRNCSL | sequence |
| | CADNIEGKTF VSTVNSLVFQ QIDANWNIQC WLKGDLKLFI | |
| | CYVESLFKNL FRNYNYKVHL LYVLPEVLED SPLVPQKGSF | |
| | QMVHCNCSVH ECCECLVPVP TAKLNDTLLM CLKITSGGVI | |
| | FQSPLMSVQP INMVKPDPPL GLHMEITDDG NLKISWSSPP | |
| | LVPFPLQYQV KYSENSTTVI READKIVSAT SLLVDSILPG | |
| | SSYEVQVRGK RLDGPGIWSD WSTPRVFTTQ DVIYFPPKIL | |
| | TSVGSNVSFH CIYKKENKIV PSKEIVWWMN LAEKIPQSQY | |
| | DVVSDHVSKV TFFNLNETKP RGKFTYDAVY CCNEHECHHR | |
| | YAELYVIDVN INISCETDGY LTKMTCRWST STIQSLAEST | |
| | LQLRYHRSSL YCSDIPSIHP ISEPKDCYLQ SDGFYECIFQ | |
| | PIFLLSGYTM WIRINHSLGS LDSPPTCVLP DSVVKPLPPS | |
| | SVKAEITINI GLLKISWEKP VFPENNLQFQ IRYGLSGKEV | |
| | QWKMYEVYDA KSKSVSLPVP DLCAVYAVQV RCKRLDGLGY | |
| | WSNWSNPAYT VVMDIKVPMR GPEFWRIING DTMKKEKNVT | |
| | LLWKPLMKND SLCSVQRYVI NHHTSCNGTW SEDVGNHTKF | |
| | TFLWTEQAHT VTVLAINSIG ASVANFNLTF SWPMSKVNIV | |
| | QSLSAYPLNS SCVIVSWILS PSDYKLMYFI IEWKNLNEDG | |
| | EIKWLRISSS VKKYYIHDHF IPIEKYQFSL YPIFMEGVGK | |
| | PKIINSFTQD DIEKHQSDEQ KLISEEDLGG EQKLISEEDL | |
| | HHHHHH | |
| 115 | FNLSYPITPW RFKLSCMPPN STYDYFLLPA GLSKNTSNSN | Amino Acid |
| | GHYETAVEPK FNSSGTHFSN LSKTTFHCCF RSEQDRNCSL | sequence |
| | CADNIEGKTF VSTVNSLVFQ QIDANWNIQC WLKGDLKLFI | |
| | CYVESLFKNL FRNYNYKVHL LYVLPEVLED SPLVPQKGSF | |
| | QMVHCNCSVH ECCECLVPVP TAKLNDTLLM CLKITSGGVI | |
| | FQSPLMSVQP INMVKPDPPL GLHMEITDDG NLKISWSSPP | |
| | LVPFPLQYQV KYSENSTTVI READKIVSAT SLLVDSILPG | |
| | SSYEVQVRGK RLDGPGIWSD WSTPRVFTTQ DVIYFPPKIL | |
| | TSVGSNVSFH CIYKKENKIV PSKEIVWWMN LAEKIPQSQY | |
| | DVVSDHVSKV TFFNLNETKP RGKFTYDAVY CCNEHECHHR | |
| | YAELYVIDVN INISCETDGY LTKMTCRWST STIQSLAEST | |
| | LQLRYHRSSL YCSDIPSIHP ISEPKDCYLQ SDGFYECIFQ | |
| | PIFLLSGYTM WIRINHSLGS LDSPPTCVLP DSVVKPLPPS | |
| | SVKAEITINI GLLKISWEKP VFPENNLQFQ IRYGLSGKEV | |
| | QWKMYEVYDA KSKSVSLPVP DLCAVYAVQV RCKRLDGLGY | |
| | WSNWSNPAYT VVMDIKVPMR GPEFWRIING DTMKKEKNVT | |
| | LLWKPLMKND SLCSVQRYVI NHHTSCNGTW SEDVGNHTKF | |
| | TFLWTEQAHT VTVLAINSIG ASVANFNLTF SWPMSKVNIV | |
| | QSLSAYPLNS SCVIVSWILS PSDYKLMYFI IEWKNLNEDG | |
| | EIKWLRISSS VKKYYIHDHF IPIEKYQFSL YPIFMEGVGK | |
| | PKIINSFTQD DIEKHQSDEP RGPTIKPCPP CKCPAPNLLG | |
| | GPSVFIFPPK IKDVLMISLS PIVTCVVVDV SEDDPDVQIS | |
| | WFVNNVEVHT AQTQTHREDY NSTLRVVSAL PIQHQDWMSG | |
| | KEFKCKVNNK DLPAPIERTI SKPKGSVRAP QVYVLPPPEE | |
| | EMTKKQVTLT CMVTDFMPED IYVEWTNNGK TELNYKNTEP | |
| | VLDSDGSYFM YSKLRVEKKN WVERNSYSCS VVHEGLHNHH | |
| | TTKSFSRTPG K | |
| 116 | FNLSYPITPW RFKLSCMPPN STYDYFLLPA GLSKNTSNSN | Amino Acid |
| | GHYETAVEPK FNSSGTHFSN LSKTTFHCCF RSEQDRNCSL | sequence |
| | CADNIEGKTF VSTVNSLVFQ QIDANWNIQC WLKGDLKLFI | |
| | CYVESLFKNL FRNYNYKVHL LYVLPEVLED SPLVPQKGSF | |
| | QMVHCNCSVH ECCECLVPVP TAKLNDTLLM CLKITSGGVI | |
| | FQSPLMSVQP INMVKPDPPL GLHMEITDDG NLKISWSSPP | |
| | LVPFPLQYQV KYSENSTTVI READKIVSAT SLLVDSILPG | |
| | SSYEVQVRGK RLDGPGIWSD WSTPRVFTTQ DVIYFPPKIL | |
| | TSVGSNVSFH CIYKKENKIV PSKEIVWWMN LAEKIPQSQY | |
| | DVVSDHVSKV TFFNLNETKP RGKFTYDAVY CCNEHECHHR | |
| | YAELYVIDVN INISCETDGY LTKMTCRWST STIQSLAEST | |
| | LQLRYHRSSL YCSDIPSIHP ISEPKDCYLQ SDGFYECIFQ | |
| | PIFLLSGYTM WIRINHSLGS LDSPPTCVLP DSVVKPLPPS | |
| | SVKAEITINI GLLKISWEKP VFPENNLQFQ IRYGLSGKEV | |
| | QWKMYEVYDA KSKSVSLPVP DLCAVYAVQV RCKRLDGLGY | |
| | WSNWSNPAYT VVMDIKVPMR GPEFWRIING DTMKKEKNVT | |
| | LLWKPLMKND SLCSVQRYVI NHHTSCNGTW SEDVGNHTKF | |
| | TFLWTEQAHT VTVLAINSIG ASVANFNLTF SWPMSKVNIV | |
| | QSLSAYPLNS SCVIVSWILS PSDYKLMYFI IEWKNLNEDG | |
| | EIKWLRISSS VKKYYIHDHF IPIEKYQFSL YPIFMEGVGK | |
| | PKIINSFTQD DIEKHQSDDK THTCPPCPAP ELLGGPSVFL | |
| | FPPKPKDTLM ISRTPEVTCV VVDVSHEDPE VKFNWYVDGV | |
| | EVHNAKTKPR EEQYNSTYRV VSVLTVLHQD WLNGKEYKCK | |
| | VSNKALPAPI EKTISKAKGQ PREPQVYTLP PSRDELTKNQ | |
| | VSLTCLVKGF YPSDIAVEWE SNGQPENNYK TTPPVLDSDG | |
| | SFFLYSKLTV DKSRWQQGNV FSCSVMHEAL HNHYTQKSLS | |
| | LSPGK | |
| 117 | FNLSYPITPW RFKLSCMPPN STYDYFLLPA GLSKNTSNLN | Amino Acid |
| | GHYETAVEFN SSDTHFSNLS KTTFHCCFRS EQDRNCSLCA | sequence |
| | DNIEGKTFVS TVNSSVFQQM GANWNIQCWL KGDLKLFICY | |
| | VESLFKNPFK NYKHKVHLLY VLPEVLEDSP LVPQKGSFQM | |
| | VHCNCSVHER CECLVPVPTA KLNDTLLMCL KITSGGVIFQ | |
| | SPLMSVQPIN MVKPDPPLGL RMEITDDGNL KISWSSPPLV | |
| | PFPLQYEVKY SENSTTVIRE ADKIVSATSL LVDGILPGSS | |
| | YEVQVRGKRL DGPGIWSDWS TPHVFTTQDV IYFPPKILTS | |
| | VGSNVSFHCI YKNENKIVSS KKIVWWMNLA EKIPQSQYDV | |
| | VSDHVSKVTF FNLNETKPRG KFTYDAVYCC NEHECHHRYA | |
| | ELYVIDVNIN ISCETDGHLT KMTCRWSTNT IQSLAGSTLQ | |
| | LRYRRSSLYC FDIPSIHPIS KPKDCYLQSD GFYECVFQPI | |
| | FLLSGYTMWI RINHPLGSLD SPPTCVLPDS VVKPLPPSSV | |
| | KAEIIKNIGL LKISWEKPVF PENNLQFQIR YGLSGKEIQW | |
| | KMYDVYDAKS KSVSLPVPDF CAVYAVQVRC KRSDGLGLWS | |
| | NWSNPAYTVV MDIKVPMRGP EFWRIINGDT MKKEKNVTLL | |
| | WKPLMKNDSL CSVQRYVINH HTSCNGTWSE DVGNHTKFTF | |
| | LWTEQAHTVT VLAINSIGAS VANFNLTFSW PMSKVNIVQS | |
| | LSAYPLNSSC VILSWILSPS DYKLMYFIIE WKNLNEDGEI | |
| | KWLRISSSVK KYYIHDHFIP IEKYQFSLYP IFMEGVGKPK | |
| | IINSFAQDNT EKHQNDEQKL ISEEDLGGEQ KLISEEDLHH HHHH | |
| 118 | LNLAYPISPW KFKLFCGPPN TTDDSFLSPA GAPNNASALK | Amino Acid |
| | GASEAIVEAK FNSSGIYVPE LSKTVFHCCF GNEQGQNCSA | sequence |
| | LTDNTEGKTL ASVVKASVFR QLGVNWDIEC WMKGDLTLFI | |
| | CHMEPLPKNP FKNYDSKVHL LYDLPEVIDD SPLPPLKDSF | |
| | QTVQCNCSLR GCECHVPVPR AKLNYALLMY LEITSAGVSF | |
| | QSPLMSLQPM LVVKPDPPLG LHMEVTDDGN LKISWDSQTM | |
| | APFPLQYQVK YLENSTIVRE AAEIVSATSL LVDSVLPGSS | |
| | YEVQVRSKRL DGSGVWSDWS SPQVFTTQDV VYFPPKILTS | |
| | VGSNASFHCI YKNENQIISS KQIVWWRNLA EKIPEIQYSI | |
| | VSDRVSKVTF SNLKATRPRG KFTYDAVYCC NEQACHHRYA | |
| | ELYVIDVNIN ISCETDGYLT KMTCRWSPST IQSLVGSTVQ | |
| | LRYHRRSLYC PDSPSIHPTS EPKNCVLQRD GFYECVFQPI | |
| | FLLSGYTMWI RINHSLGSLD SPPTCVLPDS VVKPLPPSNV | |
| | KAEITVNTGL LKVSWEKPVF PENNLQFQIR YGLSGKEIQW | |
| | KTHEVFDAKS KSASLLVSDL CAVYVVQVRC RRLDGLGYWS | |
| | NWSSPAYTLV MDVKVPMRGP EFWRKMDGDV TKKERNVTLL | |
| | WKPLTKNDSL CSVRRYVVKH RTAHNGTWSE DVGNRTNLTF | |
| | LWTEPAHTVT VLAVNSLGAS LVNFNLTFSW PMSKVSAVES | |
| | LSAYPLSSSC VILSWTLSPD DYSLLYLVIE WKILNEDDGM | |
| | KWLRIPSNVK KFYIHDNFIP IEKYQFSLYP VFMEGVGKPK | |
| | IINGFTKDAI DKQQNDAGEQ KLISEEDLGG EQKLISEEDL | |
| | HHHHHH | |
| 119 | LNLAYPTSPW RFKLFCAPPS TTDDSFLSPA GVPNNTSSLK | Amino Acid |
| | GASEALVEAK FNSTGIYVSE LSKTIFHCCF GNEQGQNCSA | sequence |
| | LTGNTEGKTL ASVVKPLVFR QLGVNWDIEC WMKGDLTLFI | |
| | CHMEPLLKNP FKNYDSKVHL LYDLPEVIDD LPLPPLKDSF | |
| | QTVQCNCSVR ECECHVPVPR AKVNYALLMY LEITSAGVSF | |
| | QSPLMSLQPM LVVKPDPPLG LRMEVTDDGN LKISWDSQTK | |
| | APFPLQYQVK YLENSTIVRE AAEIVSDTSL LVDSVLPGSS | |
| | YEVQVRSKRL DGSGVWSDWS LPQLFTTQDV MYFPPKILTS | |
| | VGSNASFCCI YKNENQTISS KQIVWWMNLA EKIPETQYNT | |
| | VSDHISKVTF SNLKATRPRG KFTYDAVYCC NEQACHHRYA | |
| | ELYVIDVNIN ISCETDGYLT KMTCRWSPST IQSLVGSTVQ | |
| | LRYHRRSLYC PDNPSIRPTS ELKNCVLQTD GFYECVFQPI | |
| | FLLSGYTMWI RINHSLGSLD SPPTCVLPDS VVKPLPPSNV | |
| | KAEITINTGL LKVSWEKPVF PENNLQFQIR YGLNGKEIQW | |
| | KTHEVFDAKS KSASLPVSDL CAVYVVQVRC RRLDGLGYWS | |
| | NWSSPAYTLV MDVKVPMRGP EFWRIMDGDI TKKERNVTLL | |
| | WKPLMKNDSL CSVRRYVVKH RTAHNGTWSQ DVGNQTNLTF | |
| | LWAESAHTVT VLAINSIGAS LVNFNLTFSW PMSKVNAVQS | |
| | LSAYPLSSSC VILSWTLSPN DYSLLYLVIE WKNLNDDDGM | |
| | KWLRIPSNVN KYYIHDNFIP IEKYQFSLYP VFMEGVGKPK | |
| | IINGFTKDDI AKQQNDAGEQ KLISEEDLGG EQKLISEEDL | |
| | HHHHHH | |
| 120 | LNLAYPISPW KFKLFCGPPN TTDDSFLSPA GAPNNASALK | Amino Acid |
| | GASEAIVEAK FNSSGIYVPE LSKTVFHCCF GNEQGQNCSA | sequence |
| | LTDNTEGKTL ASVVKASVFR QLGVNWDIEC WMKGDLTLFI | |
| | CHMEPLPKNP FKNYDSKVHL LYDLPEVIDD SPLPPLKDSF | |
| | QTVQCNCSLR GCECHVPVPR AKLNYALLMY LEITSAGVSF | |
| | QSPLMSLQPM LVVKPDPPLG LHMEVTDDGN LKISWDSQTM | |
| | APFPLQYQVK YLENSTIVRE AAEIVSATSL LVDSVLPGSS | |
| | YEVQVRSKRL DGSGVWSDWS SPQVFTTQDV VYFPPKILTS | |
| | VGSNASFHCI YKNENQIISS KQIVWWRNLA EKIPEIQYSI | |
| | VSDRVSKVTF SNLKATRPRG KFTYDAVYCC NEQACHHRYA | |
| | ELYVIDVNIN ISCETDGYLT KMTCRWSPST IQSLVGSTVQ | |
| | LRYHRRSLYC PDSPSIHPTS EPKNCVLQRD GFYECVFQPI | |
| | FLLSGYTMWI RINHSLGSLD SPPTCVLPDS VVKPLPPSNV | |
| | KAEITVNTGL LKVSWEKPVF PENNLQFQIR YGLSGKEIQW | |
| | KTHEVFDAKS KSASLLVSDL CAVYVVQVRC RRLDGLGYWS | |
| | NWSSPAYTLV MDVKVPMRGP EFWRKMDGDV TKKERNVTLL | |
| | WKPLTKNDSL CSVRRYVVKH RTAHNGTWSE DVGNRTNLTF | |
| | LWTEPAHTVT VLAVNSLGAS LVNFNLTFSW PMSKVSAVES | |
| | LSAYPLSSSC VILSWTLSPD DYSLLYLVIE WKILNEDDGM | |
| | KWLRIPSNVK KFYIHDNFIP IEKYQFSLYP VFMEGVGKPK | |
| | IINGFTKDAI DKQQNDAGDK THTCPPCPAP ELLGGPSVFL | |
| | FPPKPKDTLM ISRTPEVTCV VVDVSHEDPE VKFNWYVDGV | |
| | EVHNAKTKPR EEQYNSTYRV VSVLTVLHQD WLNGKEYKCK | |
| | VSNKALPAPI EKTISKAKGQ PREPQVYTLP PSRDELTKNQ | |
| | VSLTCLVKGF YPSDIAVEWE SNGQPENNYK TTPPVLDSDG | |
| | SFFLYSKLTV DKSRWQQGNV FSCSVMHEAL HNHYTQKSLS | |
| | LSPGK | |

What is claimed is:

1. An isolated full antibody, which is monospecific, consisting of two heavy chains and two light chains inter-connected by disulfide bonds, wherein each heavy chain comprises
a heavy chain variable region that comprises the HCDR1, HCDR2 and HCDR3 of a heavy chain variable region that comprises the amino acid sequence set forth in SEQ ID NO: 26; linked to a human heavy chain constant domain;
and
each light chain comprises
a light chain variable region that comprises the LCDR1, LCDR2 and LCDR3 of a light chain variable region that comprises the amino acid sequence set forth in SEQ ID NO: 10; linked to a human light chain constant domain.

2-24. (canceled)

25. The full antibody of claim 1 which comprises: a light chain variable region that comprises the amino acid sequence set forth in SEQ ID NO: 10; and a heavy chain variable region that comprises the amino acid sequence set forth in SEQ ID NO: 26.

26. The full antibody of claim 1 wherein the heavy chain variable region is linked to a human IgG1 heavy chain constant domain and the light chain variable region is linked to a human kappa light chain constant domain.

27. The antibody of claim 1 wherein the heavy chain variable region is linked to a human IgG4 heavy chain constant domain and the light chain variable region is linked to a human kappa light chain constant domain.

28. A pharmaceutical composition comprising the full antibody of claim 1 and a pharmaceutically acceptable carrier or diluent.

29. A pharmaceutical composition comprising the full antibody of claim 25 and a pharmaceutically acceptable carrier or diluent.

30. A pharmaceutical composition comprising the full antibody of claim 26 and a pharmaceutically acceptable carrier or diluent.

31. A pharmaceutical composition comprising the full antibody of claim 27 and a pharmaceutically acceptable carrier or diluent.

32. A pen delivery device comprising the pharmaceutical composition of claim 28.

33. A pen delivery device comprising the pharmaceutical composition of claim 29.

34. A pen delivery device comprising the pharmaceutical composition of claim 30.

35. A pen delivery device comprising the pharmaceutical composition of claim 31.

36. A method for treating a disease or condition associated with or caused by leptin deficiency or leptin resistance, the method comprising administering the pharmaceutical composition of claim 1 to a subject in need thereof.

37. The method of claim 36, wherein the disease or condition associated with or caused by leptin deficiency or leptin resistance is selected from the group consisting of lipodystrophies, obesity, metabolic syndrome, diet-induced food craving, functional hypothalamic amenorrhea, type 1 diabetes, type 2 diabetes, insulin resistance, severe insulin resistance due to mutation in insulin receptor, Alzheimer's disease, leptin deficiency, leptin resistance, Leprechaunism/Donohue syndrome, and Rabson-Mendenhall syndrome.

38. A method for treating a lipodystrophy condition in a patient, the method comprising administering the pharmaceutical composition of claim 1 to a patient in need thereof, wherein the lipodystrophy condition is selected from the group consisting of congenital generalized lipodystrophy, acquired generalized lipodystrophy, familial partial lipodystrophy, acquired partial lipodystrophy, centrifugal abdominal lipodystrophy, lipoatrophia annularis, localized lipodystrophy, and HIV-associated lipodystrophy.

39. A method for treating a disease or condition associated with or caused by a signaling-defective or signaling-impaired LEPR mutation, the method comprising administering the pharmaceutical composition of claim 1 to a subject in need thereof.

40. The method of claim 39, wherein the signaling-defective or signaling-impaired LEPR mutation is LEPR-A409E or LEPR-P316T.

41. The method of claim 39, wherein the disease or condition associated with or caused by a signaling-defective or signaling-impaired LEPR mutation is early-onset obesity.

42. The method of claim 36, further comprising administering a second therapeutic agent to the subject, wherein the second therapeutic agent is selected from the group consisting of a recombinant human leptin, a PCSK9 inhibitor, a statin, ezetimibe, insulin, an insulin variant, an insulin secretagogue, metformin, a sulfonylurea, a sodium glucose cotransporter 2 (SGLT2) Inhibitor, a GLP-1 agonist/analogue, a glucagon (GCG) inhibitor, a glucagon receptor (GCGR) inhibitor, an angiopoietin-like protein (ANGPTL) inhibitor, Phentermine, Orlistat, Topiramate, Bupropion, Topiramate/Phentermine, Bupropion/Naltrexone, Bupropion/Zonisamide, Pramlintide/Metrelepin, Lorcaserin, Cetilistat, Tesofensine, and Velneperit.


SEQUENCE LISTING (120 sequences)

SEQ ID NO: 1 - DNA, 357 bp, synthetic construct
  misc_feature 1..357: note = synthetic
  source 1..357: mol_type = other DNA
caggtgcagctggtggagtctgggggaggcgtggtccagcctgggaagtccctgagactctcctgtgtagcgtctggattcaccttcagttccgatgccatgtactgggtccgccaggctccaggcaaggggctggaatgggtggcagttatttattatgatggaaattatcaatactatgaagactccgttaagggtcgattcaccatctccagagacaattcccagaacacgctggatctgcaaatgaacagcctgagagtcgacgacacggctgtatatttctgtgcgcgtctcaactgggattactggtatctcgatctctggggccgtggcaccctggtcactgtctcctca

SEQ ID NO: 2 - protein, 119 aa, synthetic construct
  REGION 1..119: note = synthetic
QVQLVESGGGVVQPGKSLRLSCVASGFTFSSDAMYWVRQAPGKGLEWVAVIYYDGNYQYYEDSVKGRFTISRDNSQNTLDLQMNSLRVDDTAVYFCARLNWDYWYLDLWGRGTLVTVSS

SEQ ID NO: 3 - DNA, 24 bp, synthetic construct
  misc_feature 1..24: note = synthetic
  source 1..24: mol_type = other DNA
ggattcaccttcagttccgatgcc

SEQ ID NO: 4 - protein, 8 aa, synthetic construct
  REGION 1..8: note = synthetic
GFTFSSDA

SEQ ID NO: 5 - DNA, 24 bp, synthetic construct
  misc_feature 1..24: note = synthetic
  source 1..24: mol_type = other DNA
atttattatgatggaaattatcaa

SEQ ID NO: 6 - protein, 8 aa, synthetic construct
  REGION 1..8: note = synthetic
IYYDGNYQ

SEQ ID NO: 7 - DNA, 36 bp, synthetic construct
  misc_feature 1..36: note = synthetic
  source 1..36: mol_type = other DNA
gcgcgtctcaactgggattactggtatctcgatctc

SEQ ID NO: 8 - protein, 12 aa, synthetic construct
  REGION 1..12: note = synthetic
ARLNWDYWYLDL

SEQ ID NO: 9 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, synthetic construct
  misc_feature 1..18: note = synthetic
  source 1..18: mol_type = other DNA
cagagcattagcagctat

SEQ ID NO: 12 - protein, 6 aa, synthetic construct
  REGION 1..6: note = synthetic
QSISSY

SEQ ID NO: 13-14 - skipped (2 entries)

SEQ ID NO: 15 - DNA, 30 bp, synthetic construct
  misc_feature 1..30: note = synthetic
  source 1..30: mol_type = other DNA
caacagagttacagtacccctccgatcacc

SEQ ID NO: 16 - protein, 10 aa, synthetic construct
  REGION 1..10: note = synthetic
QQSYSTPPIT

SEQ ID NO: 17 - DNA, 357 bp, synthetic construct
  misc_feature 1..357: note = synthetic
  source 1..357: mol_type = other DNA
caggtgcagctggtggagtccgggggaggcgtggtccagcctgggaggtccctgagactctcctgtacagcgtctggattcaccttcagtagttatgccatgtactgggtccgccaggctccaggcaaggggctggagtgggtgtcagttatatactatgatggaagttataaatactatgcagactccgtgaagggccgattcaccatctccagagacaattccaagaacacgctgtatctgcaaatggacagcctgagagccgaggacacggctgtctattactgtgcgagttataactggaactactggtacttcgatttctggggccgtggcaccctggtcactgtctcctca

SEQ ID NO: 18 - protein, 119 aa, synthetic construct
  REGION 1..119: note = synthetic
QVQLVESGGGVVQPGRSLRLSCTASGFTFSSYAMYWVRQAPGKGLEWVSVIYYDGSYKYYADSVKGRFTISRDNSKNTLYLQMDSLRAEDTAVYYCASYNWNYWYFDFWGRGTLVTVSS

SEQ ID NO: 19 - DNA, 24 bp, synthetic construct
  misc_feature 1..24: note = synthetic
  source 1..24: mol_type = other DNA
ggattcaccttcagtagttatgcc

SEQ ID NO: 20 - protein, 8 aa, synthetic construct
  REGION 1..8: note = synthetic
GFTFSSYA

SEQ ID NO: 21 - DNA, 24 bp, synthetic construct
  misc_feature 1..24: note = synthetic
  source 1..24: mol_type = other DNA
atatactatgatggaagttataaa

SEQ ID NO: 22 - protein, 8 aa, synthetic construct
  REGION 1..8: note = synthetic
IYYDGSYK

SEQ ID NO: 23 - DNA, 36 bp, synthetic construct
  misc_feature 1..36: note = synthetic
  source 1..36: mol_type = other DNA
gcgagttataactggaactactggtacttcgatttc

SEQ ID NO: 24 - protein, 12 aa, synthetic construct
  REGION 1..12: note = synthetic
ASYNWNYWYFDF

SEQ ID NO: 25 - DNA, 357 bp, synthetic construct
  misc_feature 1..357: note = synthetic
  source 1..357: mol_type = other DNA
caggtgcagctggtggagtctgggggaagcgtggtccagcctgggaggtccctgagactctcctgtgcagcgtctggattcaccttcagtacatatgccatgtactgggtccgccagactccaggcaaggggctggagtgggtggctgttttatactctgatggaagtaataaatactatatagactccgtgaagggccgattcaccatctccagagacacttccacgaacactctgtatctgcaaatgagcagcctgcgagccgacgactcggctctatattactgtgcgcgtctcaactgggattactggtacttcgatctctggggccgtggcaccctggtcactgtctcctca

SEQ ID NO: 26 - protein, 119 aa, synthetic construct
  REGION 1..119: note = synthetic
QVQLVESGGSVVQPGRSLRLSCAASGFTFSTYAMYWVRQTPGKGLEWVAVLYSDGSNKYYIDSVKGRFTISRDTSTNTLYLQMSSLRADDSALYYCARLNWDYWYFDLWGRGTLVTVSS

SEQ ID NO: 27 - DNA, 24 bp, synthetic construct
  misc_feature 1..24: note = synthetic
  source 1..24: mol_type = other DNA
ggattcaccttcagtacatatgcc

SEQ ID NO: 28 - protein, 8 aa, synthetic construct
  REGION 1..8: note = synthetic
GFTFSTYA

SEQ ID NO: 29 - DNA, 24 bp, synthetic construct
  misc_feature 1..24: note = synthetic
  source 1..24: mol_type = other DNA
ttatactctgatggaagtaataaa

SEQ ID NO: 30 - protein, 8 aa, synthetic construct
  REGION 1..8: note = synthetic
LYSDGSNK

SEQ ID NO: 31 - DNA, 36 bp, synthetic construct
  misc_feature 1..36: note = synthetic
  source 1..36: mol_type = other DNA
gcgcgtctcaactgggattactggtacttcgatctc

SEQ ID NO: 32 - protein, 12 aa, synthetic construct
  REGION 1..12: note = synthetic
ARLNWDYWYFDL

SEQ ID NO: 33 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgaagcgtctggattcagcagcagtgacaatgccatgtactgggtccgccaggctccaggcaaggggctggagtgggtgtcagttatatatcatgatggaagttataaatactatgaagactccgtgaagggccgattcaccatcgccagagacaattccaagaacacgctttatttgcaaatgaacagcctgagagccgaggacacggctgtatattactgtgcgaggtataactggaaccactggtacttcgatgtctggggccgtggcaccctggtcactgtctcctca

SEQ ID NO: 34 - protein, 119 aa, synthetic construct
  REGION 1..119: note = synthetic
QVQLVESGGGVVQPGRSLRLSCEASGFSSSDNAMYWVRQAPGKGLEWVSVIYHDGSYKYYEDSVKGRFTIARDNSKNTLYLQMNSLRAEDTAVYYCARYNWNHWYFDVWGRGTLVTVSS

SEQ ID NO: 35 - DNA, 24 bp, synthetic construct
  misc_feature 1..24: note = synthetic
  source 1..24: mol_type = other DNA
ggattcagcagcagtgacaatgcc

SEQ ID NO: 36 - protein, 8 aa, synthetic construct
  REGION 1..8: note = synthetic
GFSSSDNA

SEQ ID NO: 37 - DNA, 24 bp, synthetic construct
  misc_feature 1..24: note = synthetic
  source 1..24: mol_type = other DNA
atatatcatgatggaagttataaa

SEQ ID NO: 38 - protein, 8 aa, synthetic construct
  REGION 1..8: note = synthetic
IYHDGSYK

SEQ ID NO: 39 - DNA, 36 bp, synthetic construct
  misc_feature 1..36: note = synthetic
  source 1..36: mol_type = other DNA
gcgaggtataactggaaccactggtacttcgatgtc

SEQ ID NO: 40 - protein, 12 aa, synthetic construct
  REGION 1..12: note = synthetic
ARYNWNHWYFDV

SEQ ID NO: 41 - DNA, 366 bp, synthetic construct
  misc_feature 1..366: note = synthetic
  source 1..366: mol_type = other DNA
caggtgcagctggtggagtctgggggaggcgtggtccagcctgggaggtccctgagactctcctgtgcagcctctggattcaccttcagtacctatggcatgcactgggtccgccaggctccaggcaaggggctggagtgggtgtcagttatatcatatgacgaaagtaataagtactatgcagactccgtgaagggccgattcaccatttctagagacaattccaagaacgcgctgtatttacaaatgaacagcctgagaaatgaggacacggctgtgtattactgtgcgagagatcggccttttggattggttaccggatggttcgacccctggggccagggaaccctggtcaccgtctcctca

SEQ ID NO: 42 - protein, 122 aa, synthetic construct
  REGION 1..122: note = synthetic
QVQLVESGGGVVQPGRSLRLSCAASGFTFSTYGMHWVRQAPGKGLEWVSVISYDESNKYYADSVKGRFTISRDNSKNALYLQMNSLRNEDTAVYYCARDRPFGLVTGWFDPWGQGTLVTVSS

SEQ ID NO: 43 - DNA, 24 bp, synthetic construct
  misc_feature 1..24: note = synthetic
  source 1..24: mol_type = other DNA
ggattcaccttcagtacctatggc

SEQ ID NO: 44 - protein, 8 aa, synthetic construct
  REGION 1..8: note = synthetic
GFTFSTYG

SEQ ID NO: 45 - DNA, 24 bp, synthetic construct
  misc_feature 1..24: note = synthetic
  source 1..24: mol_type = other DNA
atatcatatgacgaaagtaataag

SEQ ID NO: 46 - protein, 8 aa, synthetic construct
  REGION 1..8: note = synthetic
ISYDESNK

SEQ ID NO: 47 - DNA, 45 bp, synthetic construct
  misc_feature 1..45: note = synthetic
  source 1..45: mol_type = other DNA
gcgagagatcggccttttggattggttaccggatggttcgacccc

SEQ ID NO: 48 - protein, 15 aa, synthetic construct
  REGION 1..15: note = synthetic
ARDRPFGLVTGWFDP

SEQ ID NO: 49 - DNA, 360 bp, synthetic construct
  misc_feature 1..360: note = synthetic
  source 1..360: mol_type = other DNA
caggtgcagctggtggagtctgggggaggcgtggtccagcctgggaggtccctgagactctcctgtgcagcgtctggattcagtttcaatacctatggcatgcactgggtccgccaggctccaggcaaggggctggagtgggtgacaattatatggtatgatggaagtattaaatactatgcagactccgtgaagggccgattcaccatctccagagacaattccaagaacacgctgtatctgcaaatgaacagcctgagagccgaggacacggctgtgtattattgtgcgagaggtggatatagtggctacctctactttgactactggggccagggaaccctggtcaccgtctcctca

SEQ ID NO: 50 - protein, 120 aa, synthetic construct
  REGION 1..120: note = synthetic
QVQLVESGGGVVQPGRSLRLSCAASGFSFNTYGMHWVRQAPGKGLEWVTIIWYDGSIKYYADSVKGRFTISRDNSKNTLYLQMNSLRAEDTAVYYCARGGYSGYLYFDYWGQGTLVTVSS

SEQ ID NO: 51 - DNA, 24 bp, synthetic construct
  misc_feature 1..24: note = synthetic
  source 1..24: mol_type = other DNA
ggattcagtttcaatacctatggc

SEQ ID NO: 52 - protein, 8 aa, synthetic construct
  REGION 1..8: note = synthetic
GFSFNTYG

SEQ ID NO: 53 - DNA, 24 bp, synthetic construct
  misc_feature 1..24: note = synthetic
  source 1..24: mol_type = other DNA
atatggtatgatggaagtattaaa

SEQ ID NO: 54 - protein, 8 aa, synthetic construct
  REGION 1..8: note = synthetic
IWYDGSIK

SEQ ID NO: 55 - DNA, 39 bp, synthetic construct
  misc_feature 1..39: note = synthetic
  source 1..39: mol_type = other DNA
gcgagaggtggatatagtggctacctctactttgactac

SEQ ID NO: 56 - protein, 13 aa, synthetic construct
  REGION 1..13: note = synthetic
ARGGYSGYLYFDY

SEQ ID NO: 57 - DNA, 372 bp, synthetic construct
  misc_feature 1..372: note = synthetic
  source 1..372: mol_type = other DNA
caggtgcagctgcaggagtcgggcccaggactggtgaagccttcacagaccctgtccctcacctgcactgtctctggtggctccatcagcagcggtggtgactactggagctggatccgccagctcccagggaagggcctggagtggattgggtacatctattacagtgggagcgcctactataatccgtccctcaagagtcgaggtaccatatcaatagacacgtctaagaaccagttctccctgaagctgacctctgtgactgccgcggacacggccgtatatttctgtgtgaaattacgatttttggagtggttcttggggggctggttcggcccctggggccagggaaccctggtcaccgtctcctca

SEQ ID NO: 58 - protein, 124 aa, synthetic construct
  REGION 1..124: note = synthetic
QVQLQESGPGLVKPSQTLSLTCTVSGGSISSGGDYWSWIRQLPGKGLEWIGYIYYSGSAYYNPSLKSRGTISIDTSKNQFSLKLTSVTAADTAVYFCVKLRFLEWFLGGWFGPWGQGTLVTVSS

SEQ ID NO: 59 - DNA, 30 bp, synthetic construct
  misc_feature 1..30: note = synthetic
  source 1..30: mol_type = other DNA
ggtggctccatcagcagcggtggtgactac

SEQ ID NO: 60 - protein, 10 aa, synthetic construct
  REGION 1..10: note = synthetic
GGSISSGGDY

SEQ ID NO: 61 - DNA, 21 bp, synthetic construct
  misc_feature 1..21: note = synthetic
  source 1..21: mol_type = other DNA
atctattacagtgggagcgcc

SEQ ID NO: 62 - protein, 7 aa, synthetic construct
  REGION 1..7: note = synthetic
IYYSGSA

SEQ ID NO: 63 - DNA, 48 bp, synthetic construct
  misc_feature 1..48: note = synthetic
  source 1..48: mol_type = other DNA
gtgaaattacgatttttggagtggttcttggggggctggttcggcccc

SEQ ID NO: 64 - protein, 16 aa, synthetic construct
  REGION 1..16: note = synthetic
VKLRFLEWFLGGWFGP

SEQ ID NO: 65 - DNA, 351 bp, synthetic construct
  misc_feature 1..351: note = synthetic
  source 1..351: mol_type = other DNA
gaggtgcagctggtggagtctgggggaggcttggtacagccgggggggtccctgagactctcctgtgcagcctctggattcacctttagcaactatggcatgacctgggtccgccaggctccagggaagggcctggaatgggtctcagctattactggtggtggtggtagcacatactactcaaactccgtgaagggccggttcaccatctccagagacaattccaagaacacggtgtatctgcgaatgaacagtgtgagagccgaggacacggccgtatattactgtgcgaaatataagtggaacttcgtggacgactggggccagggaaccacggtcaccgtctcctca

SEQ ID NO: 66 - protein, 117 aa, synthetic construct
  REGION 1..117: note = synthetic
EVQLVESGGGLVQPGGSLRLSCAASGFTFSNYGMTWVRQAPGKGLEWVSAITGGGGSTYYSNSVKGRFTISRDNSKNTVYLRMNSVRAEDTAVYYCAKYKWNFVDDWGQGTTVTVSS

SEQ ID NO: 67 - DNA, 24 bp, synthetic construct
  misc_feature 1..24: note = synthetic
  source 1..24: mol_type = other DNA
ggattcacctttagcaactatggc

SEQ ID NO: 68 - protein, 8 aa, synthetic construct
  REGION 1..8: note = synthetic
GFTFSNYG

SEQ ID NO: 69 - DNA, 24 bp, synthetic construct
  misc_feature 1..24: note = synthetic
  source 1..24: mol_type = other DNA
attactggtggtggtggtagcaca

SEQ ID NO: 70 - protein, 8 aa, synthetic construct
  REGION 1..8: note = synthetic
ITGGGGST

SEQ ID NO: 71 - DNA, 30 bp, synthetic construct
  misc_feature 1..30: note = synthetic
  source 1..30: mol_type = other DNA
gcgaaatataagtggaacttcgtggacgac

SEQ ID NO: 72 - protein, 10 aa, synthetic construct
  REGION 1..10: note = synthetic
AKYKWNFVDD

SEQ ID NO: 73 - DNA, 360 bp, synthetic construct
  misc_feature 1..360: note = synthetic
  source 1..360: mol_type = other DNA
gaggtgcagctggtggagtctgggggaggcttggtacagcctggggggtccctgagactctcctgtgttgcctctggattcaccttcaataaatacgacatgcactgggtccgccaaactactggaaaaggtctagagtgggtctcaggtattgatactgatggtgacacatactatccaggctccgtgaagggccgattcaccatctccagagaaaatgccgagaactccctgtatcttcaaatgaacggcctgagagtcggggacacggctgtgtattactgtgcaagatggccttggagtggtttctatggtgcttttgatatctggggccaagggacaatggtcaccgtctcttca

SEQ ID NO: 74 - protein, 120 aa, synthetic construct
  REGION 1..120: note = synthetic
EVQLVESGGGLVQPGGSLRLSCVASGFTFNKYDMHWVRQTTGKGLEWVSGIDTDGDTYYPGSVKGRFTISRENAENSLYLQMNGLRVGDTAVYYCARWPWSGFYGAFDIWGQGTMVTVSS

SEQ ID NO: 75 - DNA, 24 bp, synthetic construct
  misc_feature 1..24: note = synthetic
  source 1..24: mol_type = other DNA
ggattcaccttcaataaatacgac

SEQ ID NO: 76 - protein, 8 aa, synthetic construct
  REGION 1..8: note = synthetic
GFTFNKYD

SEQ ID NO: 77 - DNA, 21 bp, synthetic construct
  misc_feature 1..21: note = synthetic
  source 1..21: mol_type = other DNA
attgatactgatggtgacaca

SEQ ID NO: 78 - protein, 7 aa, synthetic construct
  REGION 1..7: note = synthetic
IDTDGDT

SEQ ID NO: 79 - DNA, 42 bp, synthetic construct
  misc_feature 1..42: note = synthetic
  source 1..42: mol_type = other DNA
gcaagatggccttggagtggtttctatggtgcttttgatatc

SEQ ID NO: 80 - protein, 14 aa, synthetic construct
  REGION 1..14: note = synthetic
ARWPWSGFYGAFDI

SEQ ID NO: 81 - DNA, 366 bp, synthetic construct
  misc_feature 1..366: note = synthetic
  source 1..366: mol_type = other DNA
caggtgcagctgcaggagtcgggcccaggactggtgaagccttcacagaccctgtccctcacctgcactgtctctggtggctccatcagcagtggtaattactactggaactggatccgccaacagccaggagagggcctggagtggattgcttacatctatcacaatggggtcaccaacttcaatccgtccctcaagagtcgacttactatatcagtagacacgtctaagactcagttctccctgaagttgaggtctgtgactgccgcggacacggccgtttattactgtgcgagatcaggcagctggttcgagaactggtacttcgatctctggggccgtggcaccctggtcactgtctcctca

SEQ ID NO: 82 - protein, 122 aa, synthetic construct
  REGION 1..122: note = synthetic
QVQLQESGPGLVKPSQTLSLTCTVSGGSISSGNYYWNWIRQQPGEGLEWIAYIYHNGVTNFNPSLKSRLTISVDTSKTQFSLKLRSVTAADTAVYYCARSGSWFENWYFDLWGRGTLVTVSS

SEQ ID NO: 83 - DNA, 30 bp, synthetic construct
  misc_feature 1..30: note = synthetic
  source 1..30: mol_type = other DNA
ggtggctccatcagcagtggtaattactac

SEQ ID NO: 84 - protein, 10 aa, synthetic construct
  REGION 1..10: note = synthetic
GGSISSGNYY

SEQ ID NO: 85 - DNA, 21 bp, synthetic construct
  misc_feature 1..21: note = synthetic
  source 1..21: mol_type = other DNA
atctatcacaatggggtcacc

SEQ ID NO: 86 - protein, 7 aa, synthetic construct
  REGION 1..7: note = synthetic
IYHNGVT

SEQ ID NO: 87 - DNA, 42 bp, synthetic construct
  misc_feature 1..42: note = synthetic
  source 1..42: mol_type = other DNA
gcgagatcaggcagctggttcgagaactggtacttcgatctc

SEQ ID NO: 88 - protein, 14 aa, synthetic construct
  REGION 1..14: note = synthetic
ARSGSWFENWYFDL

SEQ ID NO: 89 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 90 - protein, 108 aa, synthetic construct
  REGION 1..108: note = synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 91 - DNA, 21 bp, synthetic construct
  misc_feature 1..21: note = synthetic
  source 1..21: mol_type = other DNA
cagagtgttagcagcagctac

SEQ ID NO: 92 - protein, 7 aa, synthetic construct
  REGION 1..7: note = synthetic
QSVSSSY

SEQ ID NO: 93-94 - skipped (2 entries)

SEQ ID NO: 95 - DNA, 27 bp, synthetic construct
  misc_feature 1..27: note = synthetic
  source 1..27: mol_type = other DNA
cagcagtatggtagctcaccttggacg

SEQ ID NO: 96 - protein, 9 aa, synthetic construct
  REGION 1..9: note = synthetic
QQYGSSPWT

SEQ ID NO: 97 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtggctccatcagtaattcctactggagctggatccggcagcccccagggaagggactggagtggattggatatgtctattcccgtgggaacaccaagtacaacccctccctcacgagtcgagtcaccatgtcatttgacacgtccaagaaccagttctccctgaaactgaggtctgtgaccgccgcagacacggccgtgtattactgtgcgagaagcagcagctggtacgaggactggtacttcgatctctggggccgtggcaccctggtcactgtctcctca

SEQ ID NO: 98 - protein, 120 aa, synthetic construct
  REGION 1..120: note = synthetic
QVQLQESGPGLVKPSETLSLTCTVSGGSISNSYWSWIRQPPGKGLEWIGYVYSRGNTKYNPSLTSRVTMSFDTSKNQFSLKLRSVTAADTAVYYCARSSSWYEDWYFDLWGRGTLVTVSS

SEQ ID NO: 99 - DNA, 24 bp, synthetic construct
  misc_feature 1..24: note = synthetic
  source 1..24: mol_type = other DNA
ggtggctccatcagtaattcctac

SEQ ID NO: 100 - protein, 8 aa, synthetic construct
  REGION 1..8: note = synthetic
GGSISNSY

SEQ ID NO: 101 - DNA, 21 bp, synthetic construct
  misc_feature 1..21: note = synthetic
  source 1..21: mol_type = other DNA
gtctattcccgtgggaacacc

SEQ ID NO: 102 - protein, 7 aa, synthetic construct
  REGION 1..7: note = synthetic
VYSRGNT

SEQ ID NO: 103 - DNA, 42 bp, synthetic construct
  misc_feature 1..42: note = synthetic
  source 1..42: mol_type = other DNA
gcgagaagcagcagctggtacgaggactggtacttcgatctc

SEQ ID NO: 104 - protein, 14 aa, synthetic construct
  REGION 1..14: note = synthetic
ARSSSWYEDWYFDL

SEQ ID NO: 105 - DNA, 354 bp, synthetic construct
  misc_feature 1..354: note = synthetic
  source 1..354: mol_type = other DNA
caggtgcagctacagcagtggggcgcagggctgtttaagccttcggagaccctgtccctcacctgcgatgtctatggtgggtccttcagaggttattattggagttggatccgccagcccccagggaaggggctggagtggattggggaaatcagttatagtggtttcaccaattacaacccgtccctcaagagtcgagtcatcatatcaatagatacgtccaagaaccagttctccctgaagatgagctctgtgaccgccgcggacacggctgtttattactgtgcgagagttacctatggttatgggacctttgattattggggccagggaaccctggtcaccgtctcctca

SEQ ID NO: 106 - protein, 118 aa, synthetic construct
  REGION 1..118: note = synthetic
QVQLQQWGAGLFKPSETLSLTCDVYGGSFRGYYWSWIRQPPGKGLEWIGEISYSGFTNYNPSLKSRVIISIDTSKNQFSLKMSSVTAADTAVYYCARVTYGYGTFDYWGQGTLVTVSS

SEQ ID NO: 107 - DNA, 24 bp, synthetic construct
  misc_feature 1..24: note = synthetic
  source 1..24: mol_type = other DNA
ggtgggtccttcagaggttattat

SEQ ID NO: 108 - protein, 8 aa, synthetic construct
  REGION 1..8: note = synthetic
GGSFRGYY

SEQ ID NO: 109 - DNA, 21 bp, synthetic construct
  misc_feature 1..21: note = synthetic
  source 1..21: mol_type = other DNA
atcagttatagtggtttcacc

SEQ ID NO: 110 - protein, 7 aa, synthetic construct
  REGION 1..7: note = synthetic
ISYSGFT

SEQ ID NO: 111 - DNA, 36 bp, synthetic construct
  misc_feature 1..36: note = synthetic
  source 1..36: mol_type = other DNA
gcgagagttacctatggttatgggacctttgattat

SEQ ID NO: 112 - protein, 12 aa, synthetic construct
  REGION 1..12: note = synthetic
ARVTYGYGTFDY

SEQ ID NO: 113 - protein, 1165 aa, synthetic construct
  REGION 1..1165: note = hLEPR Accession No P48357
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 114 - protein, 846 aa, synthetic construct
  REGION 1..846: note = hLEPR.mmh aa 1-818: F22-D839 of P48357 aa 819-846: myc-myc-hexahistidine tag
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDEQKLISEEDLGGEQKLISEEDLHHHHHH

SEQ ID NO: 115 - protein, 1051 aa, synthetic construct
  REGION 1..1051: note = hLEPR.mFc aa 1-818: F22-D839 of P48357 aa 819-1051: mouse IgG2a (E98-K330 of P01863)
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 116 - protein, 1045 aa, synthetic construct
  REGION 1..1045: note = hLEPR.hFc aa 1-818: F22-D839 of P48357 aa 819-1045: human IgG1 tag (D104-K330 of P01857)
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 844 aa, synthetic construct
  REGION 1..844: note = MfLEPR.mmh aa 1-816: Macaca fascicularis F22-D837 with a T827A substitution from XP_005543194.1 aa 817-844: myc-myc-hexahistidine tag
FNLSYPITPWRFKLSCMPPNSTYDYFLLPAGLSKNTSNLNGHYETAVEFNSSDTHFSNLSKTTFHCCFRSEQDRNCSLCADNIEGKTFVSTVNSSVFQQMGANWNIQCWLKGDLKLFICYVESLFKNPFKNYKHKVHLLYVLPEVLEDSPLVPQKGSFQMVHCNCSVHERCECLVPVPTAKLNDTLLMCLKITSGGVIFQSPLMSVQPINMVKPDPPLGLRMEITDDGNLKISWSSPPLVPFPLQYEVKYSENSTTVIREADKIVSATSLLVDGILPGSSYEVQVRGKRLDGPGIWSDWSTPHVFTTQDVIYFPPKILTSVGSNVSFHCIYKNENKIVSSKKIVWWMNLAEKIPQSQYDVVSDHVSKVTFFNLNETKPRGKFTYDAVYCCNEHECHHRYAELYVIDVNINISCETDGHLTKMTCRWSTNTIQSLAGSTLQLRYRRSSLYCFDIPSIHPISKPKDCYLQSDGFYECVFQPIFLLSGYTMWIRINHPLGSLDSPPTCVLPDSVVKPLPPSSVKAEIIKNIGLLKISWEKPVFPENNLQFQIRYGLSGKEIQWKMYDVYDAKSKSVSLPVPDFCAVYAVQVRCKRSDGLGLWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVILSWILSPSDYKLMYFIIEWKNLNEDGEIKWLRISSSVKKYYIHDHFIPIEKYQFSLYPIFMEGVGKPKIINSFAQDNTEKHQNDEQKLISEEDLGGEQKLISEEDLHHHHHH

SEQ ID NO: 118 - protein, 846 aa, synthetic construct
  REGION 1..846: note = mLEPR.mmh aa 1-818: Mouse LEPR (L22-G839 of NP_666258.2) aa 817-846: myc-myc-hexahistidine tag
LNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHDNFIPIEKYQFSLYPVFMEGVGKPKIINGFTKDAIDKQQNDAGEQKLISEEDLGGEQKLISEEDLHHHHHH

SEQ ID NO: 119 - protein, 846 aa, synthetic construct
  REGION 1..846: note = rLEPR.mmh aa 1-818: Rat LEPR (L22-G839 of NP_036728.1) aa 819-846: myc-myc-hexahistidine tag
LNLAYPTSPWRFKLFCAPPSTTDDSFLSPAGVPNNTSSLKGASEALVEAKFNSTGIYVSELSKTIFHCCFGNEQGQNCSALTGNTEGKTLASVVKPLVFRQLGVNWDIECWMKGDLTLFICHMEPLLKNPFKNYDSKVHLLYDLPEVIDDLPLPPLKDSFQTVQCNCSVRECECHVPVPRAKVNYALLMYLEITSAGVSFQSPLMSLQPMLVVKPDPPLGLRMEVTDDGNLKISWDSQTKAPFPLQYQVKYLENSTIVREAAEIVSDTSLLVDSVLPGSSYEVQVRSKRLDGSGVWSDWSLPQLFTTQDVMYFPPKILTSVGSNASFCCIYKNENQTISSKQIVWWMNLAEKIPETQYNTVSDHISKVTFSNLKATRPRGKFTYDAVYCCNEQACHHRYAELYVIDVNINISCETDGYLTKMTCRWSPSTIQSLVGSTVQLRYHRRSLYCPDNPSIRPTSELKNCVLQTDGFYECVFQPIFLLSGYTMWIRINHSLGSLDSPPTCVLPDSVVKPLPPSNVKAEITINTGLLKVSWEKPVFPENNLQFQIRYGLNGKEIQWKTHEVFDAKSKSASLPVSDLCAVYVVQVRCRRLDGLGYWSNWSSPAYTLVMDVKVPMRGPEFWRIMDGDITKKERNVTLLWKPLMKNDSLCSVRRYVVKHRTAHNGTWSQDVGNQTNLTFLWAESAHTVTVLAINSIGASLVNFNLTFSWPMSKVNAVQSLSAYPLSSSCVILSWTLSPNDYSLLYLVIEWKNLNDDDGMKWLRIPSNVNKYYIHDNFIPIEKYQFSLYPVFMEGVGKPKIINGFTKDDIAKQQNDAGEQKLISEEDLGGEQKLISEEDLHHHHHH

SEQ ID NO: 120 - protein, 1045 aa, synthetic construct
  REGION 1..1045: note = mLEPR.hFc aa 1-818: Mouse LEPR (L22-G839 of NP_666258.2) aa 819-1045: human IgG1 tag (D104-K330 of P01857)
LNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHDNFIPIEKYQFSLYPVFMEGVGKPKIINGFTKDAIDKQQNDAGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK